(19) United States
(12) Patent Application Publication
(10) Pub. No.: US 2026/0078335 A1
(43) Pub. Date: Mar. 19, 2026

(54) MICROBIAL ENHANCED COMPOSITIONS COMPRISING SURFACTANT BLENDS WHICH OVERCOME ANTAGONISTIC SURFACTANT INCOMPATIBILITY

(71) Applicant: Stepan Company, Northbrook, IL (US)

(72) Inventors: Sangwook Lim, Chicago, IL (US); Alexander N. Nikoloff, Wheeling, IL (US); Ronald A. Masters, Wheeling, IL (US)

(21) Appl. No.: 19/402,711

(22) Filed: Nov. 26, 2025

Related U.S. Application Data
(63) Continuation of application No. PCT/US24/31460, filed on May 29, 2024.
(60) Provisional application No. 63/469,719, filed on May 30, 2023.
(60) Provisional application No. 63/612,788, filed on Dec. 20, 2023.

(51) Int. Cl. C12N 1/20

(52) U.S. Cl. CPC C12N 1/20

(57) ABSTRACT

Disclosed are compositions that can overcome the adverse effect that particular surfactants have on the viability, growth and biological activity of live beneficial microorganisms. The compositions include at least one anionic surfactant and at least one amphoteric surfactant or glyceryl monoesters and/or diesters, each of which, individually, adversely affect viability, growth, and/or biological activity of beneficial microorganisms, but used in combination, overcome the adverse effect that each surfactant exhibits on the beneficial microorganism. The compositions further include the beneficial microorganism, and optionally, at least one nonionic surfactant that adversely affects the viability, growth, and/or biological activity of beneficial microorganism. Also disclosed is a method for overcoming the adverse effect that particular surfactants have on beneficial microorganisms by combining selected anionic surfactants and amphoteric surfactants or glyceryl monoesters and/or diesters in particular amounts and ratios with the beneficial microorganism.

### RELATED APPLICATIONS

This application is a continuation of and claims priority to PCT Application No. US2024/031460, filed May 29, 2024, which claims priority to U.S. Provisional Patent Application No. 63/612,788 having a filing date of Dec. 20, 2023, and to U.S. Provisional Patent Application No. 63/469,719, having a filing date of May 30, 2023, each of which is incorporated herein by reference in its entirety.

### BACKGROUND OF THE INVENTION

The present technology relates to compositions comprising at least two surfactants that, individually, adversely affect viability, growth, and/or biological activity of beneficial microorganisms, but in combination overcome the adverse effect of the surfactants, thereby facilitating viability, growth, and biological activity of the beneficial microorganism. The present technology also relates to a method for overcoming the adverse effect that particular surfactants have on beneficial microorganisms by combining the surfactants in particular amounts and ratios to overcome the adverse effect on the microorganisms.

There has been a recent trend to formulate products with ingredients that are based on renewable raw materials rather than fossil fuels. Such ingredients are considered “green” or “natural”, since they are derived from renewable and/or sustainable sources. As a result, they are more environmentally friendly than ingredients derived from fossil fuels. An ingredient having a high Biorenewable Carbon Index (BCI), such as greater than 80, indicates that the ingredient contains carbons that are derived primarily from plant, animal or marine-based sources.

There has also been a recent trend to formulate products using the whole cell of beneficial microorganisms, such as various Bacillus species, to enable improved results. For example, bacterial endospores (commonly referred to as spores) may be added to cleaning formulations to provide a long-term cleaning effect. Bacterial spores may also be used in agricultural applications to enhance the health and vitality of plants, and in bioremediation compositions to better promote the biodegradation of organic contaminants. When in spore form, the beneficial microorganisms do not provide any particular benefit to the composition. In order to obtain performance benefits, the spores must germinate, grow, and exert biological activity when needed. With favorable environmental conditions of pH, ionic strength, and nutrients (such as protein and oil) for food, the beneficial microorganism spores can germinate and convert into vegetative cells, at which point the microorganism produces enzymes, metabolites, digests organic materials (for example, embedded soil), solubilizes inorganic plant nutrients, and promotes a healthy microbiome, crowding out non-beneficial and pathogenic microorganisms.

An important issue for germination, growth and biological activity is the compatibility of the beneficial microorganism with other components in the formulation. It has now been found that many surfactants used in, for example, cleaning compositions and agricultural compositions, create an environment that is unfavorable for the microorganism. Surfactants that can adversely affect the viability of beneficial microorganisms include many common anionic surfactants, such as alkyl sulfates and alkyl ether sulfates, and most amphoteric surfactants. Other surfactants that adversely affect beneficial microorganisms include rhamnolipids, which are interface-active glycolipids produced by various bacterial species. Adding beneficial microorganisms to compositions containing these surfactants does not provide the desired benefits because the beneficial microorganisms fail to germinate or, after germination, fail to grow, multiply, or survive.

Another important consideration for compositions comprising beneficial microorganisms is the ability to maintain the microorganisms in a stable suspension during storage and use. Microorganisms that have settled to the bottom of the container may not be adequately dispersed with shaking or stirring, resulting in uneven or inconsistent application of the microorganisms.

There is therefore a need for compositions containing beneficial microorganisms that can maintain the viability of the microorganisms during storage and allow the beneficial microorganisms to germinate and grow during use. There is also a need for compositions that are storage stable and can maintain the microorganisms suspended without agglomeration or settling.

Applicants have determined that particular surfactants can be combined in particular amounts and ratios to overcome the adverse effects the surfactants have on the beneficial microorganism, allowing viability, growth, and biological activity of the microorganisms. Use of these surfactant blends in microbial enhanced compositions maintains the viability of the beneficial microorganisms while also advancing U.N. Sustainability Goals (“SDG”). Many bio-based surfactants are antagonistic towards beneficial microorganisms, and the particular surfactant blends described herein enable broader use of such bio-based surfactants in a variety of different applications. The bio-based surfactants are derived from renewable resources, and beneficial microorganisms, such as Bacillus species, are made by fermentation, a less energy intensive bio-based manufacturing process, using renewable resources and generating biodegradable waste products that are less impactful on the environment. These benefits further SDG #12 (Responsible Consumption and Production).

### BRIEF SUMMARY OF THE INVENTION

In one aspect, the present technology provides a composition for overcoming antagonistic surfactant incompatibility between a surfactant and a beneficial microorganism, wherein the antagonistic surfactant incompatibility adversely affects viability, growth, or biological activity of a live beneficial microorganism, the composition comprising (a) 0.1% to about 15% by active weight of at least one anionic surfactant that, individually, exhibits antagonistic surfactant incompatibility when in contact with a live beneficial microorganism and adversely affects viability, growth, or biological activity of the live beneficial microorganism, wherein the anionic surfactant is selected from the group consisting of alkyl sulfate, alkyl ether sulfate, alkyl sarcosinate, alpha sulfonated alkyl esters, alkyl glutamate, and combinations thereof; (b) 0.1% to about 15% by active weight of at least one additional surfactant that, individually, exhibits antagonistic surfactant incompatibility when in contact with a live beneficial microorganism and adversely affects viability, growth, or biological activity of the live beneficial microorganism, wherein the additional surfactant is selected from the group consisting of (i) amphoteric surfactants, and (ii) glyceryl monoesters and/or diesters; (c) optionally, least one nonionic surfactant that, individually, exhibits antagonistic surfactant incompatibility when in contact with a live beneficial microorganism and adversely affects viability, growth, or biological activity of the live beneficial microorganism; (d) at least 10⁴ CFU/mL of a beneficial microorganism; and (e) carrier to total 100% by weight of the composition, wherein the anionic surfactant and the additional surfactant, and optionally, the nonionic surfactant, in combination in the composition overcome the antagonistic surfactant incompatibility between the surfactants and the beneficial microorganism, and facilitate viability, growth, and biological activity of the beneficial microorganism.

In a further aspect, the present technology provides a method for overcoming antagonistic surfactant incompatibility between a surfactant and a beneficial microorganism, wherein the antagonistic surfactant incompatibility adversely affects viability, growth, or biological activity of a live beneficial microorganism. The method comprises (a) providing at least 10⁴ cfu/mL of a beneficial microorganism; (b) providing 0.1% to about 15% by active weight of an anionic surfactant that, individually, exhibits antagonistic surfactant incompatibility when in contact with a live beneficial microorganism and adversely affects viability, growth, or biological activity of the live beneficial microorganism, wherein the anionic surfactant is selected from the group consisting of alkyl sulfate, alkyl ether sulfate, alkyl sarcosinate, alpha sulfonated alkyl esters, alkyl glutamate, and combinations thereof; (c) providing 0.1% to about 15% by active weight of an additional surfactant that, individually, exhibits antagonistic surfactant incompatibility when in contact with a live beneficial microorganism and adversely affects viability, growth, or biological activity of the live beneficial microorganism, wherein the additional surfactant is selected from the group consisting of (i) amphoteric surfactants and (ii) glyceryl monoesters and/or diesters; (d) optionally, providing a nonionic surfactant that, individually, exhibits antagonistic surfactant incompatibility when in contact with a live beneficial microorganism and adversely affects viability, growth, or biological activity of the live beneficial microorganism; (e) mixing together the beneficial microorganism, the anionic surfactant, the additional surfactant, and optionally, the nonionic surfactant in a liquid carrier to form a composition; wherein the anionic surfactant, the additional surfactant, and optionally, the nonionic surfactant, in combination, overcome the antagonistic surfactant incompatibility between the surfactants and the beneficial microorganism, and facilitate viability, growth, and biological activity of the beneficial microorganism.

In a related aspect, the present technology provides a method of making a composition that facilitates viability, growth, and biological activity of a beneficial microorganism, wherein the method comprises (a) providing at least 10⁴ cfu/mL of a live beneficial microorganism; (b) providing 0.1% to about 15% by active weight of an anionic surfactant that, individually, exhibits antagonistic surfactant incompatibility when in contact with a live beneficial microorganism and adversely affects viability, growth, or biological activity of the live beneficial microorganism, wherein the anionic surfactant is selected from the group consisting of alkyl sulfate, alkyl ether sulfate, alkyl sarcosinate, alpha sulfonated alkyl esters, alkyl glutamate, and combinations thereof; (c) providing 0.1% to about 15% by active weight of an additional surfactant that, individually, exhibits antagonistic surfactant incompatibility when in contact with a live beneficial microorganism and adversely affects viability, growth, or biological activity of the live beneficial microorganism, wherein the additional surfactant is selected from the group consisting of (i) amphoteric surfactants and (ii) glyceryl monoesters and/or diesters; (d) optionally, providing a nonionic surfactant that, individually, exhibits antagonistic surfactant incompatibility when in contact with a live beneficial microorganism and adversely affects viability, growth, or biological activity of the live beneficial microorganism; (e) combining the beneficial microorganism, the anionic surfactant, the additional surfactant, optionally, the nonionic surfactant, and water under conditions sufficient to form the composition; whereby the anionic surfactant, the additional surfactant, and optionally, the nonionic surfactant, in combination, overcome the antagonistic surfactant incompatibility between the surfactants and the beneficial microorganism, and facilitate viability, growth, and biological activity of the beneficial microorganism.

In another aspect, the present technology provides a composition comprising about 2.0% to about 15% active weight anionic surfactant; about 2.0% to about 15% by active weight amphoteric surfactant; optionally, 0% to about 2.0% by active weight, alternatively 0.1% to about 2% by active weight, alcohol alkoxylate; optionally, 0% to about 2% by active weight, alternatively about 0.1% to about 2% by active weight, dialkyl amide or alkyl lactyl lactate; optionally, 0% to about 7.0% active weight, alternatively 0.1% to about 5% by active weight MgCl₂ or NaCl; at least 10⁴ cfu/mL of a beneficial microorganism; and water to total 100% by weight of the composition. In some embodiments, the anionic surfactant comprises alkyl ether sulfate, and the amphoteric surfactant comprises alkyl amidopropyl betaine.

In a further aspect, the present technology provides a composition comprising about 2% to about 7% by active weight alpha sulfonated alkyl ester, about 0.2% to about 2% by active weight alkyl sarcosinate, about 5% to about 15% by active weight glyceryl monoesters and/or diesters, at least 10⁴ cfu/mL of a beneficial microorganism; and water to total 100% by weight of the composition.

In a still further aspect, the present technology provides a composition comprising about 8% to about 20% by active weight, alternatively about 8% to about 15% by active weight alcohol alkoxylate having a Hydrophilic-Lipophilic Balance (HLB) value of less than 10, about 2% to about 5% by active weight alkyl amine oxide, about 0.2% to about 2% by active weight alkyl sarcosinate, at least 10⁴ cfu/mL of a beneficial microorganism; and water to total 100% by weight of the composition.

In some embodiments, the composition can be in the form of a liquid concentrate that is diluted prior to use at dilution ratios of 1:000, 1:400, 1:100, 1:64, 1:32, 1:16, or 1:10, among others. In other embodiments, the composition can be a ready to use (RTU) composition in which the active ingredients are in amounts suitable for use.

### BRIEF DESCRIPTION OF THE DRAWINGS

FIG. 1 shows the compatibility profiles of different surfactants and surfactant blends against Bacillus spores as determined using a resazurin assay.

FIG. 2 is a graph showing the effect of spore addition on the viscosity of a surfactant blend.

FIG. 3 is a contour plot showing results from viscosity testing for compositions comprising a blend of sodium lauryl ether sulfate (SLES) and cocamidopropyl betaine (CAPB).

FIG. 4 is a contour plot showing results from viscosity testing for compositions comprising a blend of SLES and CAPB and different amounts of dialkyl amide and sodium chloride.

FIG. 5 is a graph showing the dynamic viscosity of two different spore-containing compositions.

FIG. 6 is a photograph showing the stability of a representative composition of the present technology and a comparative composition.

FIG. 7 is a graph and photograph showing the sedimentation behavior over a three month period of a representative composition of the present technology.

FIG. 8 is a microscopic image showing the spore dispersity of a composition of the present technology and a comparative composition.

FIG. 9 is a graph showing the dynamic viscosity as a function of concentration for formulations containing either a dialkyl amide or alkyl lactyl lactate additive.

FIG. 10 is a contour plot showing results from viscosity testing for compositions comprising a blend of alkyl sarcosinate and alkyl betaine.

FIG. 11 is a photograph comparing the biological activity in test compositions comprising microbial spores combined with sodium lauryl sulfate alone, combined with amine oxide alone, and combined with a blend of sodium lauryl sulfate and amine oxide surfactants.

FIG. 12 is a graph showing the viscosity of a structured surfactant composition with and without bacterial spores as a function of shear rate.

FIG. 13 is a graph showing the yield stress of structured surfactant compositions having different weight ratios of low Hydrophilic-Lipophilic Balance (“HLB”) surfactants and high HLB surfactants and different amounts of bacterial spores.

### DETAILED DESCRIPTION OF THE INVENTION

### Definitions

“Beneficial microorganism” refers to a microorganism that provides positive effects on the health and well-being of living organisms and ecosystems.

“Biological activity” refers to the various processes and functions that microorganisms perform in their environment, including their ability to grow, reproduce, metabolize nutrients, release enzymes and metabolites, internalize or bind molecules, produce and respond to signals, promote nutrient cycling and availability, and interact with other organisms.

“Antagonistic surfactant incompatibility” refers to a surfactant that adversely affects the viability, growth, and biological activity of a beneficial microorganism.

“Adversely affects” or “adverse effects” refers to disruption of biological capacity (such as germination), growth potential, or biological activity of beneficial microorganisms. Such disruption includes disruption of any of the steps involved in the microorganism's life cycle, such as sporulation or germination of spores and conidia, production of metabolites and enzymes to digest food sources, metabolism of those food sources, and maintenance and/or growth of the beneficial microorganism population.

“Microbial enhanced composition” refers to a composition comprising at least one surfactant that provides primary benefits of lowered aqueous surface tension, wetting of substrates such as soil, leaves, or hard and soft surfaces, and at least one beneficial live microorganism.

“Inhibitory surfactant” refers to a surface active agent that, when used at levels which provide lowered aqueous surface tension, wetting of substrates such as soil, leaves and hard and soft surfaces, adversely affects the germination, growth, and/or biological activity of the added beneficial microorganism(s) when combined in use or in a common composition.

“Overcome surfactant incompatibility” or “overcoming surfactant incompatibility” refers to the ability of a blend of inhibitory surfactants to facilitate biological activity of beneficial microorganisms when the beneficial microorganisms are in the presence of the blend of inhibitory surfactants, as indicated by a decrease in drop size or the appearance of haze after 2-6 days, when determined in accordance with the test methods described in the Examples.

“Carbohydrates” as used herein include simple sugars and complex molecules such as but not limited to monosaccharides and disaccharides, including glucose, fructose, galactose, sucrose, lactose, maltose, xylose; polysaccharides such as starch, fiber, maltodextrin, amylose, amylopectin, glycogen; and complex sources such as molasses.

“Biorenewable Carbon Index” (BCI) refers to a calculation of the percent carbon derived from a biorenewable resource and is calculated based on the number of biorenewable carbons divided by the total number of carbons in the entire molecule.

“Biorenewable” is defined herein as originating from animal, plant, or marine material.

The terms “active”, “% active”, and “% active weight” refer to the amount of the active ingredient without regard to the amount of water or other solvent that may be present with the ingredient.

A “ready-to-use” or “RTU” product, composition or formulation of the present technology refers to a product, composition, or formulation that is ready to be applied or used as-is.

A “dilutable,” “concentrate,” or “dilutable concentrate” product, composition, or formulation of the present technology refers to a product, composition, or formulation that needs to be diluted with a diluent (e.g., water) in a ratio of, for example, 1:100, 1:64, 1:32, 1:16, or 1:10, among others, before it can be applied or used for its intended purpose.

The present technology is based on the discovery that many useful important classes of surfactants are antagonistic to beneficial microorganisms at surfactant use levels well below the levels required for typical product performance. However, it has now been found that particular surfactants that, individually exhibit antagonistic surfactant incompatibility, can be used in combination to overcome the surfactant incompatibility and render the surfactants usable in compositions that include the beneficial microorganisms. As a result, the beneficial microorganisms in the compositions are able to germinate, grow, and exert biological activity when the particular blends of these incompatible surfactants are used in particular amounts and ratios. Not to be bound by any particular theory, it is believed that the combination of particular incompatible surfactants in selected amounts and ratios provides the desired effect of overcoming the antagonistic surfactant incompatibility.

Incompatibility between a surfactant and a beneficial microorganism has been investigated in various ways, such as a standard Zone of Inhibition test, a standard agar plating test, or a standard resazurin (7-hydroxy-10-oxidophenoxazin-10-ium-3-one, sodium) (a blue fluorogenic dye) compatibility assay. In a Zone of Inhibition test, microorganisms are grown, cultured, diluted down to target levels, and then applied to a Petri dish containing agar nutrients. Test samples containing the surfactant of interest are diluted and loaded into an agar well created within the agar nutrient. The plates are incubated and microorganism growth is allowed to develop. The test sample diffuses through the agar and, if the surfactant in the test sample has an inhibitory effect on the microorganism, a lack of growth halo will be observed around the sample loaded well. This halo is referred to as the zone of inhibition. The larger the zone, the bigger the effect of inhibition. Major suitability limitations for non-antibiotics test samples for this test method are inherent large dilution of active, agar diffusion challenges, and nutrient media incompatibilities (neutralization, precipitation, etc). Although this method has been used in the medical field for antibiotics very successfully for decades, it has low suitability when testing non-antibiotic samples. In a standard agar plating test, test samples containing the surfactant of interest and microorganisms are exposed, diluted several orders of magnitude, and plated onto a nutritive agar that is incubated. After an incubation period appropriate for the species, the microorganism growth pattern is evaluated against appropriate controls. This method is suitable for evaluation of live counts over time, however, not very suitable for testing of inhibition due to the significant serial dilutions that remove all coformulants from the microbial species tested on the evaluative agar plate. In a resazurin-based test, test samples containing the surfactant of interest are mixed with reduced microbial nutrients, the beneficial microorganism, and resazurin in liquid suspension. The test samples are incubated and then inspected for colorometric and fluorescence change. Test samples that are blue and have low fluorescence indicate no biological activity and incompatibility between the surfactant and the microorganism. Test samples that are pink and have high fluorescence indicate biological activity and compatibility of the surfactant with the microorganism. This test and other similar liquid suspension tests address the previously mentioned traditional test limitations and provide a suitable, microorganism agnostic, high-throughput platform to evaluate inhibitive effects of surfactants with biologicals.

Surfactants that have been found to be incompatible with beneficial microorganisms at surfactant concentrations of 1 g/L or less include some anionic surfactants, some non-ionic surfactants, most, if not all, amphoteric surfactants, and most, if not all, cationic surfactants. Specific incompatible surfactants include alkyl sulfates, alkyl ether sulfates, alkyl sarcosinates, alkyl glutamates, alpha sulfonated alkyl esters, alkyl sulfonates, alkyl sulfoacetates, some alkyl phosphate esters, sulfosuccinates, rhamnolipids, hydroxy sultaines, alkyl betaines, alkyl amidopropyl betaines, alkyl amine oxides, alkyl amine alkoxylates, quaternized alkyl amine alkoxylates, some alcohol alkoxylates, mono- and/or diglycerides, and some EO/PO block copolymers. Surprisingly, however, blending together selected incompatible surfactants in selected amounts and ratios can produce a surfactant blend that is compatible with beneficial microorganisms, thereby enabling the incompatible surfactants to be used in compositions employing the beneficial microorganisms. The blend of incompatible surfactants comprises at least one incompatible anionic surfactant and at least one additional surfactant that is incompatible with a beneficial microorganism. The additional incompatible surfactant may be an amphoteric surfactant, or glyceryl monoesters and/or diesters, or a combination thereof. The blend can optionally include at least one nonionic surfactant that is incompatible with a beneficial microorganism.

The anionic surfactants that can be used in the blend of surfactants include alkyl sulfates, alkyl ether sulfates, alpha sulfonated alkyl esters, alkyl sarcosinates, and alkyl glutamates, including sodium, potassium, magnesium, ammonium, monoethanolammonium, diethanolammonium, or triethanolammonium salts thereof. Preferably, the anionic surfactants are derived from a natural source, and have a BCI of at least 80, alternatively at least 90, alternatively at least 95, and preferably 100. Examples of incompatible anionic surfactants for use in the compositions of the present technology are sodium lauroyl sulfate, sodium lauryl ether sulfates, particularly those having one, two, or three moles of ethylene oxide, sodium sulfonated methyl C₁₂-C₁₈ ester (mono) and disodium sulfonated C₁₂-C₁₈ fatty acid (di), sodium lauroyl sarcosinate, and sodium cocoyl glutamate. Combinations of incompatible anionic surfactants may also be used. In one embodiment, the compositions of the present technology can comprise a combination of alpha sulfonated alkyl esters and alkyl sarcosinates as the anionic surfactant component. The compositions of the present technology can comprise at least 0.1 wt % active anionic surfactant, such as 0.1 wt % active to about 15 wt % active. In some embodiments, the amount of anionic surfactant can be in the range of about 2 wt % active to about 15 wt % active, alternatively about 2 wt % active to about 10 wt % active.

The amphoteric surfactants that can be used in the blend of surfactants include alkyl amine oxides, alkyl betaines, alkyl amidopropyl betaines and alkyl sultaines. Preferably, the amphoteric surfactants are derived from a natural source, and have a BCI of at least 80, alternatively at least 90, alternatively at least 95, and preferably 100. Examples of incompatible amphoteric surfactants include lauramine oxide, cetyl betaine, cocoamidopropyl betaine, and cocoamidopropyl hydroxysultaine. The compositions of the present technology can comprise from 0% to about 15 wt % active of amphoteric surfactant. When the composition comprises an amphoteric surfactant, the amount of amphoteric surfactant is at least 0.1 wt % active amphoteric surfactant, such as 0.1 wt % to about 15 wt %. In some embodiments, the amount of amphoteric surfactant can be in the range of about 1 wt % active to about 15 wt % active, alternatively about 1 wt % to about 10 wt %, alternatively about 2 wt % to about 10 wt % active.

The incompatible nonionic surfactants that can be used in the blend of surfactants include alcohol alkoxylates, preferably alcohol ethoxylates, or glyceryl esters. The alcohol ethoxylates can be linear or branched, and can have HLB values from about 5 to about 15. Specific examples of incompatible alcohol ethoxylates include BIOSOFT® EC 639, a linear alcohol ethoxylate with 8 moles of ethylene oxide, BIOSOFT® N23-3, a C12-C13 semi-linear alcohol ethoxylate with 3 moles of ethylene oxide, BIOSOFT® N1-7, a C11 semi-linear alcohol ethoxylate with 7 moles of ethylene oxide, MAKON® DA-6, a C10 branched alcohol ethoxylate with 6 moles of ethylene oxide, MAKON® UD-5, a C11 branched alcohol ethoxylate with 5 moles of ethylene oxide, and MAKON® DA-4, a C10 branched alcohol ethoxylate with 4 moles of ethylene oxide, all available from Stepan Company, Northfield, Illinois. The glyceryl esters can be mono-glycerides and/or di-glycerides, or combinations thereof. A specific example of an incompatible glycerides surfactant is STEPAN-MILD® GCC, a glyceryl caprylate/caprate mono- and di-glycerides surfactant. The amount of nonionic surfactant in the compositions can be in the range of 0% to about 15 wt % active, alternatively 0% to about 13 wt % active.

Specific blends of incompatible anionic surfactants and incompatible amphoteric surfactants that have been found to be compatible with beneficial microorganisms include a blend of alkyl ether sulfate with alkyl amidopropyl betaine and/or alkyl betaine, a blend of alkyl sulfate with alkyl amine oxide, and a blend of alkyl sarcosinate with alkyl betaine. The weight ratio of anionic surfactant to amphoteric surfactant in the blend can be in the range of 5:1 to 1:2, based on actives level. Specific blends of incompatible anionic surfactants and incompatible nonionic surfactants include a blend of alpha sulfonated alkyl esters with glyceryl alkyl esters in a weight ratio of nonionic surfactant to anionic surfactant in the range of about 2:1 to about 2.5:1, based on actives level.

The total amount of surfactant in the compositions will vary depending on whether the composition is a concentrate that is intended to be diluted before use with water or other diluent, or whether the composition is a ready-to-use composition in which the active components are already at end-use concentrations. Total surfactant amounts in a concentrate composition may be in the range of about 3% to about 20% by active weight, based on the total weight of the composition. For a ready-to-use composition, the surfactant amounts may be in the range of 0.1% to about 1% by active weight, based on the total weight of the composition.

In addition to overcoming antagonistic surfactant incompatibility and facilitating the viability, growth, and biological activity of beneficial microorganisms, the surfactant blends in the compositions of the present technology unexpectedly provide improved stability and dispersibility of the beneficial microorganisms in the compositions, and prevent or minimize spore agglomeration. Stability as used herein refers not just to the ability of the compositions to maintain spore viability without premature germination during storage, but also the ability of the compositions to maintain the beneficial microorganisms, particularly microbial spores, in suspension without settling. It has been found that incorporating a beneficial microorganism into a surfactant composition can significantly reduce the viscosity of the composition, resulting in microbial enhanced compositions that are unable to keep the microorganism in a stable suspension. As a result, the compositions are unable to deliver the desired enhanced product performance that would be expected from the addition of the beneficial microorganism.

Surprisingly, compositions of the present technology provide an improved viscosity profile that enables the beneficial microorganism to remain suspended without settling. A viscosity profile for the composition can be determined by measuring viscosity as a function of shear rate at ambient temperature. A preferred viscosity profile is one in which the composition has a high viscosity at low shear rate to suspend the spores in the composition, and a reduced viscosity at high shear rate to facilitate handling of the composition. Compositions of the present technology desirably have a viscosity of about 8,000 cP to about 30,000 cP as measured using a viscometer at 6 rpm and ambient temperature (about 25° C.), and about 3,000 cP to about 8,000 cP as measured using a viscometer at 60 rpm and ambient temperature. Compositions of the present technology have viscosities within both of these low shear and high shear ranges, and can maintain the spores in suspension without significant separation, sedimentation, or precipitation for at least one month, alternatively at least two months, preferably at least three months, and more preferably at least six months. The improved stability is at least partially due to the ability of the surfactant blends described herein to build viscosity and maintain the spores in dispersion without spore agglomeration or aggregation.

Physical stability of the compositions can be assessed using different methods known in the art. For the dilutable concentrate, the shelf life can be assessed by using a Turbiscan (multiple light scattering) method along with the average size of microbial spores as a function of storage time. For ready-to-use (RTU), the dispersity of microbial spores in the composition can be assessed by using a dynamic light scattering method along with the average diameter and quantitative size distribution (polydispersity) of microbial spores. The formation of spore agglomeration or aggregation can be monitored by optical microscope with qualitative size distribution of the spores for both dilutable concentrate and ready-to-use (RTU) compositions.

In some embodiments, the surfactant blends can form a structured surfactant system that enables the spores to remain in suspension. The term “structured system” or “structured surfactant system” as used herein means a pourable composition comprising water, surfactant, and optionally other dissolved matter, which together form a mesophase, or a dispersion of a mesophase in a continuous aqueous medium, and which has the ability to immobilize non-colloidal, water-insoluble particles, while the system is at rest, thereby forming a stable, pourable suspension. Surfactants and water interact to form phases that are neither liquids nor crystals; these are usually termed “liquid crystal phases,” or alternatively “mesomorphic phases” or “mesophases.” Structured surfactant systems are generally known in the art, and are described in further detail, for example, in U.S. Pat. No. 9,668,474, incorporated herein by reference. The surfactants that can be used for preparing the structured surfactant system typically comprise a mixture of at least one surfactant having an HLB value that is low, for example less than 10, alternatively, less than 9, and at least one surfactant having an HLB value that is high, for example 10 or greater. Specific surfactant blends that can both form structured surfactant systems that suspend microbial spores in a stable suspension and overcome antagonistic surfactant incompatibility include alpha sulfonated alkyl esters combined with glyceryl caprylate/caprate esters and alkyl sarcosinates, and alkyl amine oxides combined with low HLB ethoxylated fatty alcohols and alkyl sarcosinates.

In some embodiments, it may be desirable to include an inorganic salt and/or certain dialkyl amides or certain alkyl lactyl lactates in the compositions of the present technology. It has been found that adding certain inorganic salts and/or a suitable dialkyl amide or alkyl lactyl lactate, in particular amounts, can increase the viscosity of the compositions and improve the stability and dispersibility of the beneficial microorganisms in the composition. Suitable inorganic salts include sodium chloride and magnesium chloride (or a hydrate thereof). One example of a suitable dialkyl amide is NINOL®CAA, a mixture of dimethyl lauramide and dimethyl myristamide (“CAA”) available from Stepan Company. CAA is derived primarily from renewable sources, and has a BCI of 86. One example of a suitable alkyl lactyl lactate is STEPAN-MILD® L3 (“L3”), a lauryl lactyl lactate having a BCI of 100, available from Stepan Company. When added, the amounts of inorganic salt, dialkyl amide, or alkyl lactyl lactate in the composition will depend upon the particular surfactants and the particular surfactant amounts in the composition. The amount of sodium chloride or magnesium chloride in the compositions can range from 0% to 7% by weight, alternatively about 1% to about 5% by weight of the composition. The amount of dialkyl amides or alkyl lactyl lactates in the compositions can range from 0% to about 2 wt % active, alternatively about 0.1 wt % to about 2 wt % active, based on the total weight of the composition. A weight ratio of anionic and amphoteric surfactants to dialkyl amides or alkyl lactyl lactates can be in the range of about 10:1 to about 25:1, preferably in the range of about 15:1 to about 25:1. In one embodiment, a composition having improved stability and dispersibility and a good viscosity profile comprises about 2.0% to about 15% active weight alkyl ether sulfate anionic surfactant, about 2.0% to about 15% by active weight alkylamidopropyl betaine amphoteric surfactant, 0% to about 2% by active weight, alternatively about 0.1% to about 2% by active weight alcohol alkoxylate nonionic surfactant, 0% to about 2% by active weight, alternatively about 0.1% to about 2% by active weight dialkyl amide or alkyl lactyl lactate, 0% to about 7.0%, alternatively about 1% to about 5% by weight MgCl₂ or NaCl, at least 1×10⁴ CFU/g beneficial microorganisms, and water to total 100% by weight of the composition.

The beneficial microorganisms that can be used in the compositions of the present technology can be bacteria, fungi, yeast, or mold, and can be endospores (also referred to as spores), vegetative, conidia, or mycelium cells. In some embodiments, the bacterial endospores are of the Bacillus genus. The Bacillus spores can be one or more of Bacillus amyloliquefaciens, Bacillus brevis, non-pathogenic variants of Bacillus cereus, such as toyoi, Bacillus circulans, Bacillus clausii, Bacillus coagulans, Bacillus firmus, Bacillus halodurans, Bacillus lentus, Bacillus licheniformis, Bacillus megaterium, Bacillus methylotrophicus, Bacillus mycoides, Bacillus pasteurii, Bacillus, polyfermenticus, Bacillus polymyxa, Bacillus pumilus, Bacillus simplex, Bacillus sphaericus, Bacillus stearothermophilus, Bacillus subtilis, Bacillus thiaminolyticus, Bacillus thuringiensis, and combinations thereof. In some embodiments, the beneficial microorganisms are blends of bacterial spores of two or more strains, particularly blends of two or more Bacillus strains. Blends of Bacillus spores of different Bacillus species are commercially available from different sources. Blends of Bacillus spores may include spores from one or more of Bacillus subtilis, Bacillus amyloliquefaciens, Bacillus licheniformis, Bacillus megaterium, Bacillus thuringiensis, and Bacillus pumilus. The amount of the beneficial microorganisms in the compositions of the present technology will depend on the end use for the composition, but will generally be at least 1×10⁴ CFU/g, more preferably at least 1×10⁵ CFU/g. When the composition is a concentrate that will be diluted prior to use, the amount of beneficial microorganisms can be in the range of 1×10⁷ to 1×10¹⁰ CFU/g, preferably 1×10⁹ to 5×10⁹ CFU/g. When the composition is a ready-to-use composition, the amount of the beneficial microorganism can be in the range of 1×10⁴ to 1×10⁹, alternatively 1×10⁵ to 1×10⁹ CFU/g.

The compositions of the present technology can also include additives that can enhance the ability of the surfactant blend to facilitate viability, growth, and metabolic activity of the beneficial microorganism. The additives can comprise one or more of L-amino acid, inorganic divalent metal salts, or monovalent salts. The additives may also include a carbohydrate as an optional component.

The L-amino acid can be one or more L-amino acids selected from the group consisting of L-histidine, L-isoleucine, L-leucine, L-lysine, L-methionine, L-phenylalanine, L-threonine, L-tryptophan, L-valine, L-alanine, L-asparagine, L-aspartic acid, L-glutamic acid, L-serine, L-arginine, L-cysteine, L-glutamine, L-glycine, L-proline, and L-tyrosine. In some embodiments, the L-amino acid is L-alanine.

The inorganic divalent metal salt can be one or more of magnesium chloride, calcium chloride, manganese chloride, iron chloride, copper chloride, zinc chloride, cobalt chloride, magnesium nitrate, magnesium sulfate, calcium sulfate, manganese sulfate, iron sulfate, copper sulfate, zinc sulfate, colbalt sulfate, magnesium citrate, calcium citrate, hydrates of any of the foregoing, or combinations of any of the foregoing. In some embodiments, the divalent metal salt is magnesium chloride or a hydrate thereof.

The monovalent salt can be one or more of potassium nitrate, sodium nitrate, potassium chloride, potassium iodide, potassium manganese oxide, potassium sulfate, sodium bicarbonate, sodium sulfate, ammonium nitrate, ammonium sulfate, ammonium chloride, sodium citrate, potassium citrate, ammonium citrate, or combinations thereof. In some embodiments, the monovalent metal salt is potassium nitrate.

The additives can optionally include a carbohydrate, which may provide a food source for the beneficial microorganism and enhance the extent of growth of the microorganism. The carbohydrate can be one or more of glucose, maltose, galactose, fructose, sucrose, lactose, molasses, glycogen, or glucans. In some embodiments, the carbohydrate is glucose. If a carbohydrate is included in the additive, the amount is preferably not greater than the total weight of the other additive components (L-amino acid, divalent metal salt and/or monovalent salt) in the composition.

The compositions of the present technology can include additional optional ingredients depending on end use of the compositions. Such other ingredients can include additional surfactants that are compatible with the beneficial microorganisms, hydrotropic or other solubilizing agents for obtaining and maintaining a clear single-phase concentrate or ready-to-use composition, carbohydrates as a supplemental food source for the beneficial microorganisms, builders, pH adjustment agents, electrolytes for enhancement of surfactant detergency, enzymes for cleaning enhancement, fragrances for different attractive smells, dyes for pleasing color, preservatives, and other functional ingredients. Preferably, any optional ingredients are compatible with the beneficial microorganism.

Surfactants that have been found to be compatible with the beneficial microorganisms and can be included in the composition include, but are not limited to, castor oil ethoxylates, alpha olefin sulfonates, such as BIO-TERGER AS-40, a C14-16 olefin sulfonate, polyoxyethylene sorbitan monooleates, such as those sold under the tradenames TWEEN® and SPAN®, certain alkyl dimethyl amides, such as STEPOSOL® MET-10U, an unsaturated C10 N, N-dimethyl amide, and combinations thereof.

Hydrotropes suitable for use in the compositions of the present technology include sodium xylene sulfonate, cumene sulfonate, amphoteric dipropionate salt, and combinations thereof. Suitable carbohydrates for use as a supplemental food source include cellulose, maltodextrin, fiber, amylose, amylopectin, glycogen, starch, or combinations thereof. Builders that have been found to be compatible with beneficial microorganisms include sodium gluconate and sodium citrate dihydrate.

Enzymes can be included in the compositions of the present technology to enhance cleaning. Suitable enzymes include proteases, amylases, and lipases.

Preservatives may be included in the compositions of the present technology, provided the amount of the preservative is at a level that does not affect the viability, growth, and/or biological activity of the beneficial microorganism at use dilutions of the composition. Determining an appropriate amount of preservative to be used in the compositions requires a careful balance between obtaining good efficacy against pathogenic bacteria and fungi and maintaining the viability of the beneficial microorganism in the concentrated state, and allowing beneficial microorganism growth in the ready to use state. Not all preservatives are able to achieve this balance.

For example, 2-phenoxyethanol is a common preservative used in hard surface cleaner compositions. A typical concentration for 2-phenoxyethanol is about 0.2 wt % of the composition, and an amount of 1.5 wt % is usually sufficient to preserve most compositions. However, in some embodiments of the present technology, 2-phenoxyethanol at a concentration of 1.5 wt % may not provide sufficient efficacy against pathogenic bacteria and fungi. Combining benzoic acid with 2-phenoxyethanol at a concentration of 1.5 wt % (0.75% 2-phenoxyethanol and 0.75% benzoic acid) provides good efficacy against pathogenic bacteria and fungi without affecting the viability, growth, and/or biological activity of the beneficial microorganism. Other preservatives that have been found to provide a balance between good efficacy against pathogenic bacteria and fungi and maintaining viability and performance of the beneficial microorganism include a combination of chloromethylisothiazolinone and methyl isothiazolinone (for example Kathon CG), benzisothiazolinone, and benzoic acid. Other preservatives that could be considered for use include benzyl alcohol and fatty alcohols. Combinations of any of these preservatives could also be considered. The amount of the preservative will vary depending at least in part on the particular preservative or combination of preservatives selected, but in general, it is desirable to use the lowest amount of preservative that provides the required efficacy against pathogenic bacteria and fungi.

The compositions of the present technology are typically in liquid form, and comprise at least one carrier to bring the total percentage of the composition to 100%. Water is a suitable carrier, particularly for a Ready-to-Use formulation, and can be de-ionized water, hard water, soft water, distilled water, tap water or combinations thereof. Water can be used alone as the carrier, or in combination with other suitable carriers, such as for example, water-miscible solvents, such as alcohols or glycol ethers, provided such solvents are compatible with the beneficial microorganism.

The compositions of the present technology can be used for a variety of end uses. In particular, it is contemplated that the compositions of the present technology could be used for any end use that employs a beneficial microorganism and at least one anionic incompatible surfactant and at least one amphoteric incompatible surfactant, where the combination of surfactants overcomes the antagonistic surfactant incompatibility. Such end uses can include microbial enhanced cleaners, including detergents for hard and soft surfaces, agricultural formulations, personal care formulations, bioremediation, wastewater treatment, fermentation, probiotics, animal health, aquaculture, water reclamation, and food applications.

As one example, the compositions of the present technology can be used as a microbial enhanced cleaning composition. Upon use of the composition for cleaning surfaces, the spores germinate and digest soils that are often not accessible during initial cleaning, such as soils embedded in porous surfaces such as grout, floors, and counters. The surfactant blend in the cleaning composition provides initial cleaning of the surfaces, while the combination of surfactants overcomes the antagonistic surfactant incompatibility that each surfactant exhibits individually, thereby enabling viability and growth of the microbial spores to provide enhanced cleaning. It is envisaged that the microbial enhanced cleaning compositions employing the surfactant blends described herein could be formulated without the microbial spores, which can be added to the composition at the time of use. Alternatively, the microbial enhanced compositions could be formulated as a complete microbial enhanced cleaning composition comprising the microbial spores and the combination of the surfactants, along with any optional ingredients.

The microbial enhanced cleaning compositions can be formulated, for example, as a ready-to-use product or dilutable concentrate product. A concentrated product may be up to a factor of 100× the ready-to-use component levels. Whether in a ready-to-use form or a dilutable concentrate, the end use concentrations of the components are equivalent. End use concentrations can comprise about 0.05 wt % to about 1 wt % surfactant active, alternatively about 0.1 wt % to about 1 wt % surfactant active, and about 1×10⁴ to about 1×10⁸ CFU/g alternatively about 1×10⁵ to about 1×10⁸ CFU/g beneficial microorganisms. Dilutable concentrates may comprise about 3.0 wt % to about 20 wt % surfactant active, alternatively about 5 wt % to about 15 wt % surfactant active, and about 1×10⁷ to about 1×10¹⁰ CFU/g beneficial microorganisms. In some embodiments, dilutable compositions are preferred as a cost saving and money saving option, which reduces packaging and shipping cost. In some embodiments, the concentrate may be diluted to the working concentration on site and packaged as a ready to use liquid or spray.

The diluent for diluting the concentrate form of the composition can be any diluent system known in the art. Examples of suitable diluents include, but are not limited to, water, glycols (preferably propylene glycol), alcohols (e.g., isopropanol, ethanol, methanol), other polar solvents known in the art, and mixtures thereof. Water is a preferred diluent of the presently described technology, and can be de-ionized water, hard water, soft water, distilled water, tap water or combinations thereof.

As another example of end use, the compositions of the present technology can be used in agricultural bio-pesticide and bio-fungicide formulations that employ inhibitory anionic and amphoteric surfactants in combination with beneficial microorganisms. In agriculture, microorganisms, typically of the spore type and typically of the Bacillus species, are applied to plants and/or fields to help limit growth of pathogenic organisms including fungi, leading to healthier growing crops. Many agricultural applications rely on surfactants to effectively wet leafy surfaces for efficient use of the ingredients carried by the formulation, including microorganisms like Bacillus. However, these agricultural surfactants can adversely affect viability, growth, and/or biological activity of the microorganisms. The particular blends of anionic surfactant and amphoteric surfactant in the compositions of the present technology can overcome the adverse effects of each surfactant, thereby enabling use of these inhibitory surfactants in agricultural bio-pesticide and bio-fungicide formulations.

For agricultural applications, the compositions of the present technology are typically formulated as a concentrate, and diluted with water or other suitable diluent prior to use at a dilution ratio in the range of 1:2.5 to 1:3200, alternatively 1:50 to 1:1500, alternatively 1:100 to 1:1000, alternatively 1:256 to 1:512, and preferably a dilution ratio of 1:400. The concentrate may comprise about 1.0 wt % to about 20 wt % surfactant active, and about 1×10⁷ to about 1×10¹⁰ CFU/g beneficial microorganisms.

The present technology also includes a method for overcoming surfactant incompatibility between a live beneficial microorganism and a surfactant that adversely affects the viability, growth or biological activity of the live beneficial microorganism. The method involves mixing the live beneficial microorganism with a blend of at least one anionic surfactant that exhibits the antagonistic surfactant incompatibility and at least one amphoteric surfactant or glyceryl monoesters and/or diesters surfactant that exhibits antagonistic surfactant incompatibility in a liquid carrier, such as water, wherein the combination of the anionic surfactant and amphoteric or glyceryl esters surfactant overcomes the antagonistic surfactant incompatibility and facilitates viability, growth, and biological activity of the beneficial microorganism. The amount of anionic surfactant is in the range of 0.1% to 15% by active weight, and the amount of amphoteric of glyceryl monoesters and/or diesters is in the range of 0.1% to 15% by active weight. The weight ratio of anionic surfactant to amphoteric or glyceryl esters surfactant in the blend can be in the range of 5:1 to 1:2 based on actives level. In some embodiments, the method may further comprise adding a nonionic surfactant that exhibits antagonistic surfactant incompatibility to the anionic surfactant and amphoteric surfactant. In one embodiment of the method, the beneficial microorganism is mixed with the incompatible anionic surfactant (and the nonionic surfactant, if present) in the liquid carrier, preferably water, and the incompatible amphoteric surfactant is then added and mixed to build viscosity of the composition. In another embodiment, when the surfactant amounts are at use dilution in the liquid carrier and viscosity of the end composition may be water-thin, the beneficial microorganism may be added after mixing the surfactants together to minimize settling of the beneficial microorganism out of solution. In a further embodiment, the incompatible anionic surfactant and the incompatible glyceryl monoesters and/or diesters are mixed in water in a first step to form a structured surfactant system, and the beneficial microorganism in water is added to the structured surfactant system in a second step.

The pH of the composition can be adjusted to a pH in the range of 5.5 to 8 if necessary, depending on the selection of the incompatible surfactants. Standard mixing equipment can be used to mix the incompatible surfactants and beneficial microorganism.

One skilled in the art will recognize that modifications may be made in the present technology without deviating from the spirit or scope of the invention. The invention is further illustrated by the following examples, which are not to be construed as limiting the invention in spirit or scope to the specific procedures or compositions described therein.

The pH of the compositions was determined at room temperature (20-25° C.) using a calibrated electrode.

Stat-Ease 360 software was used to generate each design of experiment sample group, model the test results from the resulting data, and generate statistics for the optimal models. The models were then used to generate an optimization plot using Stat-Ease 360's Numerical Optimization function. The optimization includes a contour plot of “desirability” of component levels for the defined design space. These plots visually represent the complex relationships between the surfactants and any additional components and show where desired performance is high and where it is low or zero.

A resazurin-based assay was used in some examples to determine compatibility of bacteria spores with a surfactant or a blend of surfactants. The assay was performed by combining the surfactant or blend with bacteria spores, limited microbial base nutrients, and resazurin in water, and incubating the test sample. The test sample is then inspected for colorimetric and fluorescence change. A test sample that is blue and has low fluorescence indicates no biological activity, and an incompatibility between the surfactant and the microorganism. Test samples that are pink and have high fluorescence indicate biological activity and compatibility of the surfactant with the microorganism.

### EXAMPLES

### Example 1: Compatibility Testing of Surfactants and Surfactant Blends

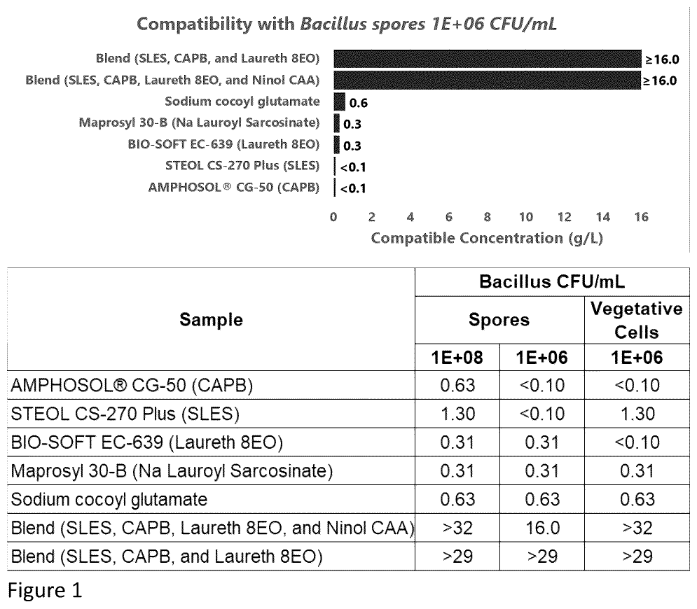

Compatibility between a beneficial microorganism and different surfactants and surfactant blends was determined using the resazurin-based assay. Aqueous test compositions were prepared and comprised the surfactant of interest in different concentrations, along with limited nutrient base media, a blend of Bacillus bacteria spores, and resazurin dye in water. A surfactant is considered inhibitory if the test composition containing a surfactant concentration of less than 1.0 g/L remains blue and shows no fluorescence when compared against appropriate controls. The surfactants tested included alkyl ether sulfate (STEOL® CS-270 Plus, sodium lauryl ether sulfate 2EO (2 moles of ethylene oxide)) (“SLES”), alkyl amidopropyl betaine (AMPHOSOL® CG-50, cocoamidopropyl betaine) (“CAPB”), alkyl sarcosinate (MAPROSYL 30-B, sodium lauroyl sarcosinate), alkyl glutamate (sodium cocoyl glutamate), and alcohol ethoxylate (BIO-SOFT® EC-639, lauryl alcohol with 8 moles of ethylene oxide) (“laureth 8EO”). Two surfactant blends were also tested: a blend of SLES, CAPB, and laureth 8EO, and a blend of SLES, CAPB, laureth 8EO, and alkyl dimethyl amide (NINOL®CAA) (“CAA”). The results of the resazurin assay are shown in FIG. 1. Short bars indicate the surfactant is inhibitory and is incompatible with the Bacillus spores, long bars indicate the surfactant is compatible with the spores. As shown in FIG. 1, each of the individual surfactants was found to be inhibitory. Surprisingly, the blend of SLES, CAPB, and laureth 8EO shows compatibility with the Bacillus spores at a high surfactant concentration (above 10 g/L) even though each of SLES, CAPB, and laureth 8EO, individually, is inhibitory at a concentration of less than 1 g/L. Similarly, the blend of SLES, CAPB, laureth 8EO, and CAA is compatible with the spores at a concentration above 10 g/L, even though each of SLES, CAPB, and laureth 8EO, individually, is inhibitory. The results show that particular blends of inhibitory surfactants are able to overcome the otherwise inhibitory action on the Bacillus spores each surfactant exhibits.

### Example 2: Effect of Spore Addition on Composition Viscosity

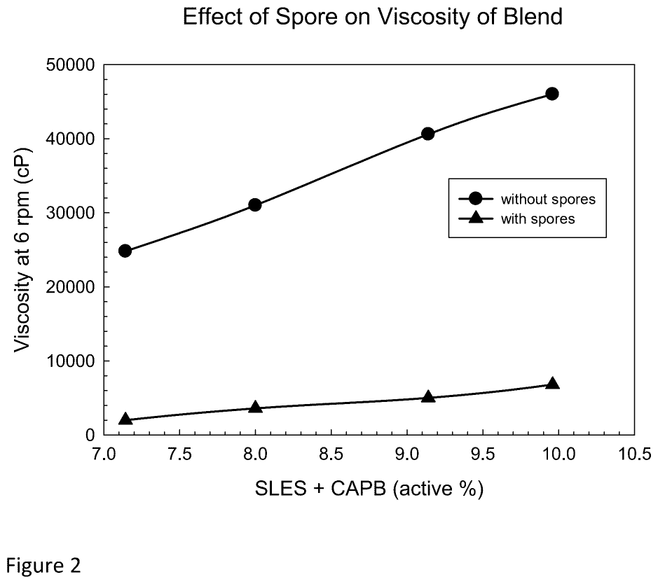

Aqueous test compositions were prepared comprising a blend of SLES anionic surfactant and CAPB amphoteric surfactant using different amounts of the surfactant blend. Each composition also contained 4.5% by weight sodium chloride. Similar compositions were also prepared except each composition also included 2×10⁹ CFU/g of a Bacillus spore blend available from commercial sources. Dynamic viscosity of the compositions was measured at 6 rpm and ambient temperature. The viscosity data was plotted as a function of surfactant concentrations, and the results are graphically shown in FIG. 2. The FIG. 2 graph shows that, without the spores, the compositions have viscosities of about 25,000 cP to about 46,000 cP, depending on surfactant concentration. With the addition of the spores, however, the compositions have viscosities of only about 2,000 cP to about 6,000 cP, at the same surfactant concentrations. The results show that the addition of a beneficial microorganism can significantly reduce the viscosity of a surfactant composition.

### Example 3: Composition with Alkyl Ether Sulfate and Alkyl Amidopropyl Betaine Surfactants

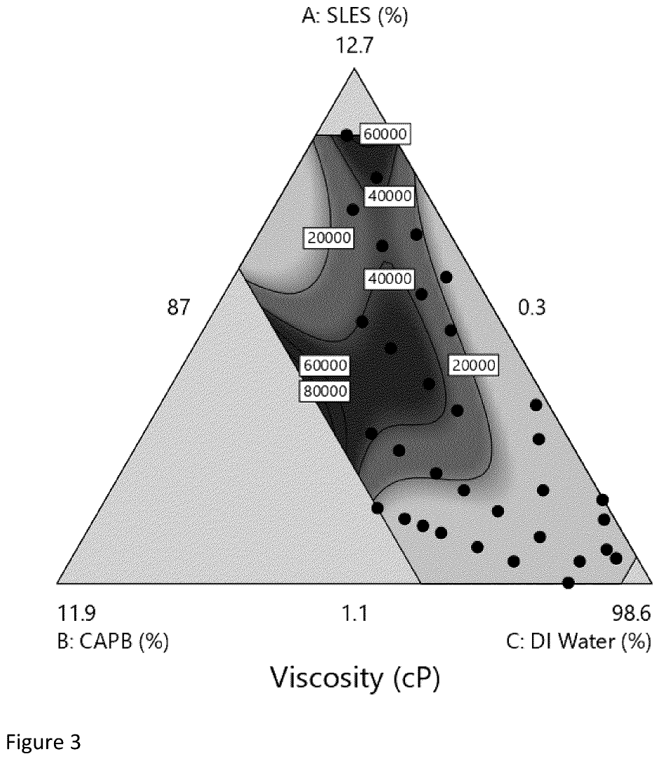

Compositions were prepared using a blend of alkyl ether sulfate (sodium lauryl ether sulfate 2EO) (“SLES”) anionic surfactant and alkyl amidopropyl betaine (cocamidopropyl betaine) (“CAPB”) amphoteric surfactant using different amounts of SLES and CAPB. All compositions contained 4.5 wt % NaCl and 1×10⁶ CFU/g of a Bacillus spore blend available from commercial sources. The dynamic viscosity of each composition was measured using a viscometer at 6 rpm at ambient temperature. The viscosity data was used to generate a Stat-Ease 360 optimization contour plot for the test compositions. The contour plot is shown in FIG. 3. The contour plot shows contour lines that depict areas of desirable high viscosity (20000 cP or more) (central area of plot) and areas of less desirable low viscosity (lower right corner and upper left area of plot) corresponding to different amounts of SLES and CAPB. From the contour plot, one can determine that the central area of the plot indicates a composition where high physical stability (a stable suspension of spores) is expected. As shown in FIG. 3, the areas of high viscosity occur with particular amounts and weight ratios of SLES and CAPB. A useful amount of SLES for obtaining a high viscosity may be in the range of about 2.75% to about 11.5% by active weight, and a useful amount of CAPB may be in the range of about 2% to about 6% by active weight. A weight ratio of SLES to CAPB can be in the range of about 20:1 to 1:1, alternatively 10:1 to 1:1, alternatively 5:1 to 1:1, alternatively 3.0:1 to 1:1, and preferably in the range of about 2.7:1 to about 1:1.

### Example 4: Composition with Alkyl Ether Sulfate and Alkyl Amidopropyl Betaine Surfactants and Optional Components

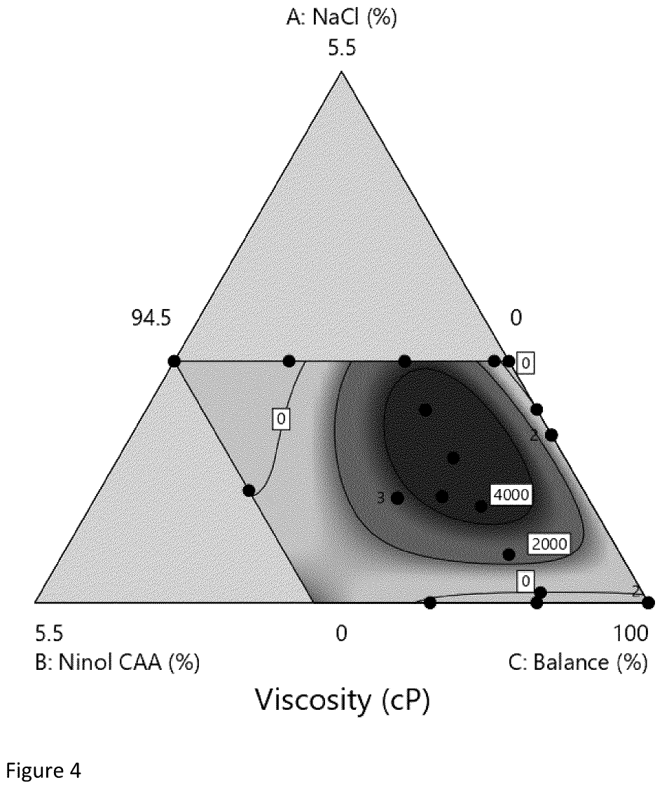

Test compositions comprising SLES and CAPB surfactants and different amounts of NaCl and alkyl dimethyl amide, NINOL®CAA, (“CAA”), were prepared to evaluate whether the addition of NaCl and/or CAA can increase the viscosity of the SLES/CAPB surfactant blend further, especially at lower surfactant concentrations. The viscosity of the test compositions was determined and used to generate a Stat-Ease 360 optimization contour plot for the test compositions. The contour plot is shown in FIG. 4. The optimization contour plot shows contour lines that depict areas of high desirability (central to right side of plot) and areas of low to zero desirability (left side, lower right corner, and far upper right side of plot) corresponding to different amounts of NaCl and CAA in the compositions. The areas of high desirability show that maximum viscosity occurs with a combination of CAA and NaCl, but that the amount of CAA should not exceed 2.5 wt %. The distinct regions of high to zero desirability show that adding NaCl and CAA to the SLES and CAPB surfactant blend can increase the viscosity of the composition, and further show that the particular amounts of the components are important for achieving the viscosity needed for maintaining physical stability of the beneficial microorganism in the composition.

### Example 5: Viscosity Profile of Composition with Alkyl Ether Sulfate, Alkyl Amidopropyl Betaine, and Optional Components

A microbial enhanced composition was prepared using SLES as the anionic surfactant, and CAPB as the amphoteric surfactant, and a commercially available Bacillus spore blend as the beneficial microorganism. The formulation also included CAA, NaCl, and a nonionic surfactant, BIOSOFT EC 639, an alcohol ethoxylate having 8 moles of ethoxylate per molecule, that is incompatible with a beneficial microorganism. Two compositions were prepared and the composition formulations are shown in Table 1.

TABLE 1

| | Composition A | | Composition B | |
|---|---|---|---|---|
| Component | wt % | % active | wt % | % active |
| DI Water | 78.65 | | 79.0 | |
| SLES 70%, | 9.6 | 6.72 | 7.6 | 5.32 |
| low 1,4 doxane | | | | |
| (STEOL CS-270C) | | | | |
| CAPB, 37%, NaCl 8% | 7 | 2.59 | 5.1 | 1.887 |
| (AMPHOSOL CG-50) | | | | |
| LAE-8, 90% | 0.6 | 0.54 | 0.8 | 0.72 |
| (BIOSOFT EC639) | | | | |
| Dimethyl Lauramide/ | 0.45 | 0.45 | 1.5 | 1.5 |
| Myristamide, | | | | |
| 100% (NINOL CAA) | | | | |
| Bacillus spores 10% | 3.7 | 1.85E+09 | 5 | 2.50E+09 |
| (Bacillus spore blend) | | CFU/g | | CFU/g |
| Sodium Chloride (NaCl) | 0 | 3.89 | 1 | 5.908 |
| Lactic Acid or | q.s pH to | | q.s pH to | |
| citric acid | 6.0-7.0 | | 6.0-7.0 | |
| | 100 | | 100 | |

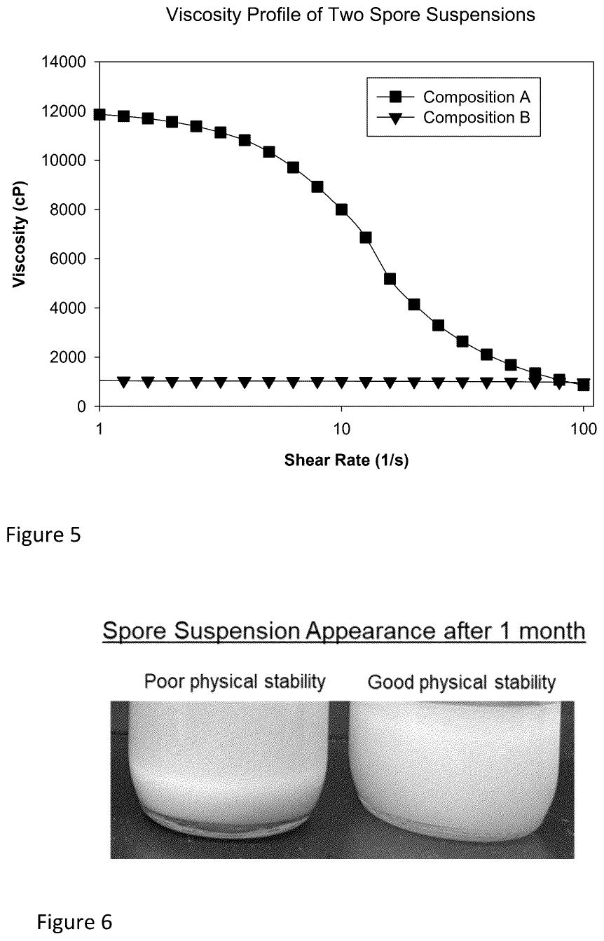

Viscosity of the two compositions was measured by using a rheometer as a function of shear rate at 25° C. FIG. 5 shows the viscosity profile of the two compositions as a function of shear rate. Although both compositions show similar viscosity, around 1000 cP, at high shear rate, Composition A provides high viscosity, around 12000 cP, at low shear, meaning the composition can suspend the spores when the composition is at rest, but the composition is also flowable at high shear rate. Composition B, however, provides low viscosity, around 1000 cP, at even a low shear rate, indicating the composition is not adequate to suspend the spores.

FIG. 6 shows the appearance of Compositions A and B after 1 month, stored at ambient temperature. Composition A shows good physical stability without settling whereas Composition B shows noticeable settling on the bottom of the glass container. The results in FIGS. 5 and 6 show that the particular amounts and ratios of the components are important for achieving the desired viscosity and stability properties. Here, the amounts of the SLES and CAPB surfactants in Composition B are not sufficient to completely solubilize the CAA, resulting in separation and low viscosity. The component amounts in Composition B are near the low desirability regions in the contour plots generated in Examples 3 and 4. Increasing the amount of CAPB, or adjusting the amount of CAA and/or NaCl in Composition B are ways to adjust the component amounts and ratios to achieve desirable viscosity and stability properties.

Composition A has the following physical properties: specific gravity: 1.03-1.05 g/cm³, active content via refractive index, Brix scale: 13.0-18.0, pH as is: 6.2-7.7, viscosity as measured using a viscometer at 25 C, 60 rpm: 4000-8000 cP.

### Example 6: Spore Dispersity in Composition with Alkyl Ether Sulfate, Alkyl Amidopropyl Betaine, and Nonionic Surfactants

Composition A in EXAMPLE 5 was evaluated for dispersity properties. Turbiscan was used to determine the ability of Composition A to maintain dispersity of the spores over time without sedimentation. Over a three month period of storage at ambient temperature, Composition A exhibited good physical stability without settling. FIG. 5 graphically shows the Turbiscan dispersity results and photographically shows the ability of Composition A to maintain physical stability without settling. The Turbiscan results demonstrate that Composition A of the present technology is physically stable and can suspend bacterial spores over time without sedimentation.

Composition A is a concentrate and was diluted with DI water at 1:100 to provide a RTU composition. The number of spores in the RTU composition is around 1.9×10⁷ CFU/g. The dispersity of the spores in the RTU composition was then characterized by dynamic light scattering. A spore suspension (1.9×10⁷ CFU/g) without the surfactants was also prepared with DI water as a comparison. Table 2 shows the dispersity of these two compositions characterized by average size, polydispersity, and particle count.

TABLE 2

| Sample | Z-average | PDI | Count |
|---|---|---|---|
| RTU formulation of | 1301 nm | 0.05 | 284 |
| Composition A | | | |
| Spore suspension with | 2020 nm | 0.23 | 204 |
| DI water (Control) | | | |

Table 2 shows the average size of spores in the RTU formulation was less than the control and the polydispersity was 4 times lower than the control. The particle count in the RTU formulation was also higher than the one in the control. The higher average spore size and polydispersity, and the lower particle count of the control suspension indicates the formation of spore aggregates. The results in Table 2 demonstrate that the RTU formulation of Composition A of the present technology provides good dispersity of bacterial spores at a RTU dilution level without spore aggregation.

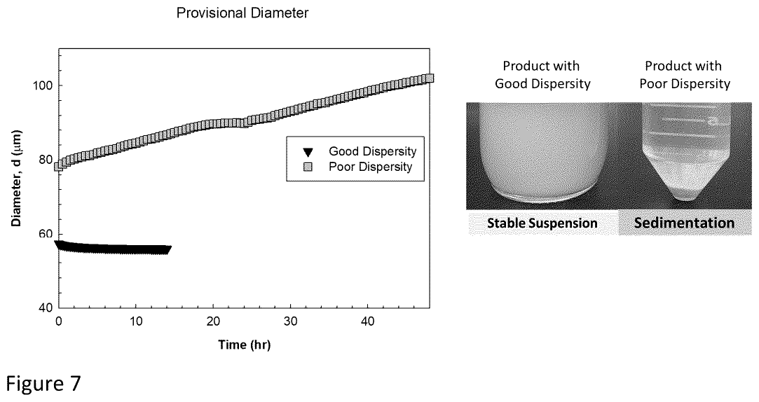

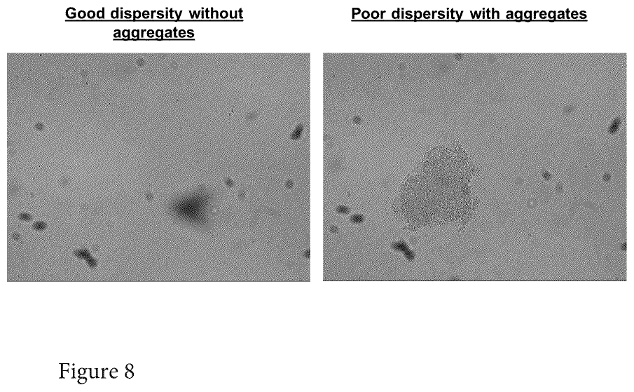

A microscopic image analysis of the RTU formulation was conducted to see the appearance of the spores in the RTU formulation. The objective with ×40 magnification was used to view the particles above 1 micrometer. FIG. 8 shows the appearance of the spores under the optical microscope. The image is compared to the control suspensions comprising the spores without the surfactant blend of Composition A. The RTU formulation shows no aggregates of spores whereas large aggregates of spores are observed in the spore suspension without the surfactant blend. This observation is consistent with the dynamic light scattering results shown in FIG. 7. Overall, the results demonstrate that Composition A of the present technology provides good dispersity of bacterial spores at both a concentrate level and a RTU level.

### Example 7: Composition Replacing Alkyl Dimethyl Amide with Alkyl Lactyl Lactate

This example evaluated whether alkyl lactyl lactate (STEPANMILD® L3) could replace dialkyl amide (dimethyl lauramide/myristamide) in a formulation similar to Example 5 Composition A and obtain similar viscosity and stability properties. Test compositions were prepared using the formulation shown in Table 3, with one set of replicate test compositions containing dialkyl amide, and a second set of replicate test compositions containing alkyl lactyl lactate.

TABLE 3
Test Formulation

| | Amount | Actives |
|---|---|---|
| Component | (wt %) | Amount (wt %) |
| DI water | 77.9% | |
| SLES (low 1,4-dioxane | 9.6% | 6.72% |
| 70%) | | |
| CAPB (37%, NaCl 13%) | 8.2% | 3.04% |
| LAE-8 (90%) | 0.4% | 0.36% |
| Dimethyl | 0.5% | 0.5% |
| lauramide/myristamide or | | |
| Lauryl lactyl lactate | | |
| Bacillus spore blend | 3.4% | 2.5E+09 |
| | | CFU/g |
| Lactic acid or | q.s. pH | |
| Citric acid | 6.5-7.0 | |

The test compositions were prepared by adding 1.0 wt % of the Table 3 formulation to water, with dialkyl amide used for one set of test compositions and alkyl lactyl lactate used for the other set. Each test composition also contained 1.0 wt % of 33 g/L Nutrient Mix (30 g/L Tryptic Soy broth and 3 g/L Yeast Extract).

The test compositions were visually evaluated for germination and growth of the bacterial spores using a Sudan Ill dyed extra virgin olive oil (EVOO) test. EVOO is a food source for vegetative cells, and can be an indicator of biological activity when added to a test sample containing bacterial spores. Under favorable conditions, bacterial spores germinate and grow into fully functional vegetative cells that secrete enzymes that can digest the EVOO. Growth and biological activity of the microorganism is evident from a thinner, more intense red layer on top of the test sample due to digestion of the EVOO and an increased concentration of the undigested Sudan Ill dye. Growth and biological activity is also evident from haze that develops in the test composition, which can also be visually detected. Test compositions that do not develop haze indicate no or minimal biological activity for the microorganism. For the dyed EVOO test, 0.2 wt % of Sudan III (62.5 ppm) dyed EVOO was added to each test composition, and the pH was adjusted to pH of 7 if needed, using L-lactic acid or dilute NaOH. The test compositions were initially photographed, and then photographed after 4 days of orbital-mixer shaking at setting “5” on a scale of 0-10. The formulations containing dialkyl amide and alkyl lactyl lactate demonstrated good biological growth, indicating that the surfactant blends in the formulations were able to overcome the incompatibility between each individual surfactant and the bacterial spores.

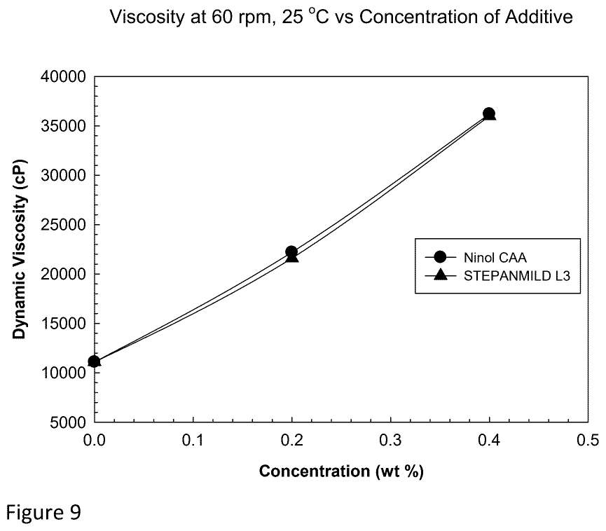

The dynamic viscosity of the dialkyl amide formulation test compositions and the alkyl lactyl lactate formulation test compositions was measured at 6 rpm and ambient temperature. The viscosity data was plotted as a function of the dialkyl amide and alkyl lactyl lactate concentrations in the respective compositions and is shown in FIG. 9. The graph in FIG. 9 shows that the viscosities of the two formulations are equivalent, demonstrating that alkyl lactyl lactate can be a 1 to 1 replacement for the dialkyl amide for increasing the viscosity of the compositions and improving the stability and dispersibility of the beneficial microorganisms in the composition.

### Example 8: Composition with Alkyl Sarcosinate and Alkyl Betaine Surfactants and Optional Components

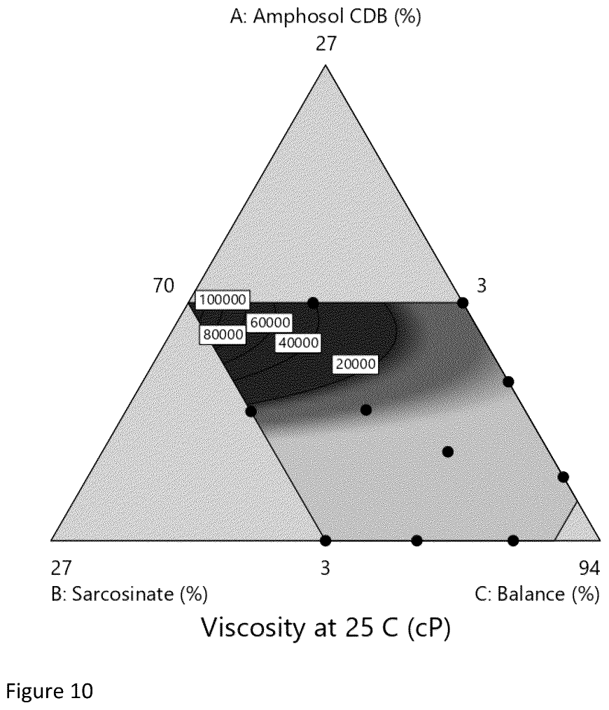

Aqueous compositions were prepared comprising a blend of alkyl sarcosinate (sodium lauroyl sarcosinate) anionic surfactant and alkyl betaine (cetyl betaine) amphoteric surfactant at different amounts and ratios. All compositions contained 1×10⁶ CFU/g of a commercially available Bacillus spore blend. The dynamic viscosity of the compositions was measured using a viscometer at 6 rpm, ambient temperature. The viscosity data was used to generate a Stat-Ease 360 optimization contour plot for the test compositions. The contour plot is shown in FIG. 10. The contour plot shows contour lines that depict areas of high viscosity (20000 cP or more) (left corner of plot) and areas of low viscosity (right corner of plot) corresponding to different amounts of alkyl sarcosinate and cetyl betaine. From the contour plot, one can determine that the area in the left corner of the plot indicates a composition where high physical stability (stable suspension of spores) is expected. As shown in FIG. 10, the areas of high viscosity occur with particular amounts and weight ratios of the alkyl sarcosinate and alkyl betaine surfactants. A preferred weight ratio of alkyl sarcosinate to alkyl betaine is about 1:1 to about 1:2.

Replicate test compositions were prepared to evaluate the ability of the alkyl sarcosinate and alkyl betaine surfactant blend to overcome the antagonistic incompatibility between each surfactant individually and the bacterial spores. The formulation for testing comprised 20 wt % sodium lauroyl sarcosinate (30% actives), 32 wt % cetyl betaine (30% actives), 3.5 wt % of a commercially available Bacillus spore blend containing 100×10⁹ CFU/g Bacillus spores, 4 wt % potassium nitrate, lactic acid (88% active) to adjust the pH to 5.5-6.0, and the remainder water. The test compositions were prepared by diluting the test formulation in water to 0.3% strength, and adding 2 wt % of 3.3 g/L Nutrient Mix (3 g/L Tryptic Soy broth and 0.3 g/L Yeast Extract). Germination and growth of bacterial spores were visually evaluated using an un-dyed EVOO test. As explained above, EVOO is a food source for vegetative cells, and can be an indicator of biological activity when added to a test sample containing bacterial spores. Under favorable conditions, bacterial spores germinate and grow into fully functional vegetative cells that secrete enzymes that can digest the EVOO. Growth and biological activity of the microorganism is evident from haze that develops in the test composition, which can be visually detected. Test compositions that do not develop haze indicate no or minimal biological activity for the microorganism. For the un-dyed EVOO test, 0.1 wt % un-dyed EVOO was added to each test composition. The compositions were then photographed initially, and re-photographed after 6.5 days of orbital-mixer shaking at setting “5” on a scale of 0-10, and the initial and aged compositions were compared visually to determine differences. All of the test compositions showed haze after 6.5 days, indicating that the surfactant blend in the test composition was able to overcome the incompatibility between each individual surfactant and the bacterial spores.

### Example 9: Test of Sodium Lauryl Sulfate, Lauramine Oxide and Surfactant Blend

Bacterial spores were added to each of three test compositions, with one test composition comprising an aqueous solution of sodium lauryl sulfate anionic surfactant (STEPANOL® WA-Extra HP), one test composition comprising an aqueous solution of lauramine oxide surfactant (AMMONYX® LO from Stepan Company), and one test composition comprising an aqueous solution of a blend of the two surfactants, to evaluate the ability of the surfactant blend to overcome the antagonistic incompatibility between each surfactant individually and the bacterial spores. Each test composition comprised 1×10⁷ CFU/g of a commercially available Bacillus spore blend and surfactant in an amount of 0.2 wt % active in water. The test composition comprising the surfactant blend comprised 0.1 wt % active of each surfactant for a total of 0.2 wt % active surfactant. Each test composition also contained 2 wt % of 3.3 g/L Nutrient Mix (3 g/L Tryptic Soy broth and 0.3 g/L Yeast Extract), and the pH of the test composition was adjusted to a pH of 7 if needed, using L-lactic acid or dilute NaOH.

The test compositions were visually evaluated for germination and growth of the bacterial spores using the un-dyed EVOO test described above, except that the test compositions were photographed after 5 days of orbital-mixer shaking at setting “5”.

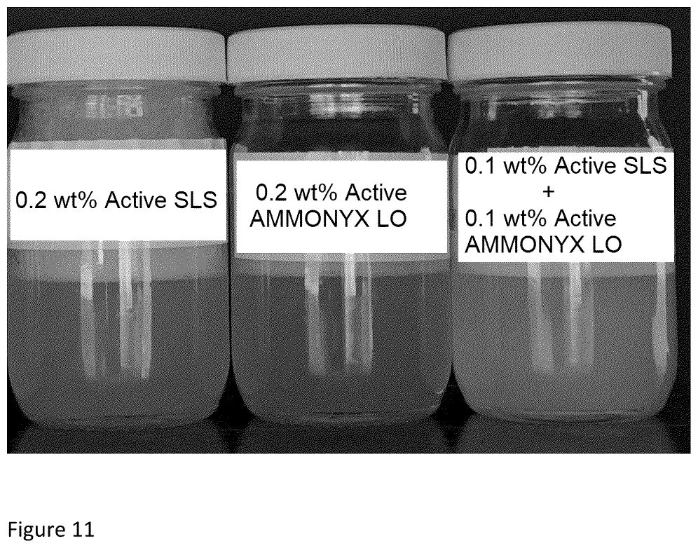

FIG. 11 is a photograph showing the test composition jars. The jar on the left is the test composition comprising only sodium lauryl sulfate, the jar in the middle is the test composition comprising only lauramine oxide, and the jar on the right is the test composition comprising the blend of the two surfactants. As shown in the photograph, the test composition comprising the blend of sodium lauryl sulfate and lauramine oxide surfactants had significant haze due to biological growth in the test composition, whereas the test compositions containing the individual surfactants showed strong inhibition of the Bacillus, being much clearer with visual haze similar to that of the initial Bacillus population only. The blend of the two surfactants is able to overcome the otherwise inhibitory action on the Bacillus that each surfactant exhibits.

### Example 10: Structured Surfactant System Containing Anionic and Nonionic Surfactants

Aqueous test compositions were prepared comprising a blend of an incompatible low-HLB nonionic surfactant with an incompatible high-HLB surfactant to evaluate the ability of the surfactant blend to form a structured aqueous system that can suspend bacterial spores. The test compositions comprised glyceryl caprylate/caprate mono- and di-esters (STEPANMILD® GCC) (“GCC”) as the low-HLB surfactant and alpha-sulfonated alkyl esters (ALPHA-STEP PC-48) (“PC-48”) as an anionic high HLB surfactant. Each test composition comprised GCC and PC-48 at a total actives concentration of about 15.5 wt %, but the relative actives weight ratio of the two surfactants was varied between 1:3 to 3:1. Each test composition also comprised 1 wt % alkyl sarcosinate surfactant (Maprosyl® 30-B) (0.3% actives), 0.2 wt % 2-phenoxyethanol, 2.68×10⁹ CFU/ml of a commercially available Bacillus spore blend, and water to total 100% by weight of the composition.

The test compositions were prepared by combining the GCC and PC-48 in water in a first stage, and then adding the alkyl sarcosinate, Bacillus spores, 2-phenoxyethanol and water in a second stage. The test compositions were visually inspected for the ability to form a structured surfactant system that can suspend the bacterial spores. The formation of a structured surfactant system can be visually determined by observing suspended air bubbles throughout the system, increased viscosity, and stability as a homogeneous solution. Testing showed that actives weight ratios of GCC to PC-48 in the range of about 2:1 to about 2.5:1 GCC to PC-48 can form structured systems that can suspend Bacillus spores, but an actives weight ratio of 3:1 GCC to PC-48 showed immediate separation.

A test composition containing an actives weight ratio of GCC to PC-48 of about 2.3:1 was selected for evaluating the effect of varying spore concentration on the stability of the formulation. The test compositions contained a total actives concentration of GCC and PC-48 of about 15.5 wt %, 1 wt % alkyl sarcosinate (0.30% actives), 0.2 wt % 2-phenoxyethanol, and water, but the spore concentration was varied. The spore concentrations tested were 0 wt %, 1.8 wt %, 2.5 wt %, and 5 wt % of a commercially available Bacillus spore blend containing 1.0×10¹¹ CFU/ml spores. The actives weight ratio of GCC to total anionic surfactant (PC-48 plus alkyl sarcosinate) in the compositions was about 2.2:1. The test compositions comprising 0 wt %, 1.8 wt %, and 2.5 wt % of the Bacillus spore blend formed stable structured surfactant systems that suspended the spores. The test composition comprising 5 wt % of the Bacillus spore blend could not maintain a stable suspension and separated into layers.

### Example 11: Rheological Properties of Structured System Containing Spores

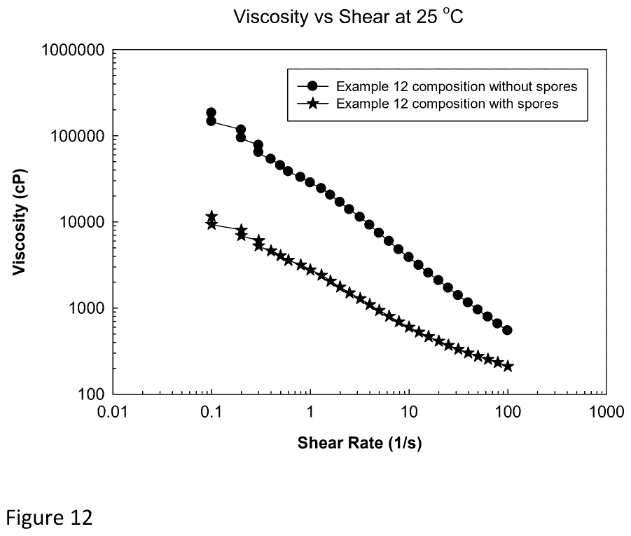

The test compositions from Example 10 having a GCC to PC-48 actives weight ratio of about 2.3:1, and containing spore concentrations of 0 wt % spores and 2.5 wt % of the Bacillus spore blend, were tested to determine the effect of bacterial spores on the rheological properties of the composition. The viscosity of the test compositions as a function of shear rate was measured by a rheometer at 25° C. The graph in FIG. 12 shows the viscosity testing results. As shown in FIG. 12, the test composition containing 2.5 wt % of the Bacillus spore blend has a dramatically decreased viscosity compared to the test composition without spores for all ranges of shear rate from 0.1/s to 100/s. However, the structuring provided by the combination of surfactants in the composition with spores still maintains a relatively high viscosity (about 10,000 cP) at low shear rate. Under high shear, the viscosity drops to about 100 cP, allowing ease of handling of the composition. The test composition without the spores exhibits a relatively high viscosity, even under high shear, indicating it is more difficult to handle than the composition with spores.

The viscosity of the test compositions was also measured as a function of temperature, varying the temperature from 5° C. to 60° C. at a fixed 60/s shear rate. Both test compositions (with and without spores) provide a fairly consistent viscosity over the temperature range, especially at temperatures above 20° C., although overall, the viscosity of the composition with spores is lower than the composition without spores.

### Example 12 Compatibility of Structured Surfactant Blend and Spores

Aqueous test compositions were prepared from a test formulation comprising glyceryl caprylate/caprate mono- and di-esters (STEPANMILD® GCC) (“GCC”) and alpha-sulfonated alkyl esters (ALPHA-STEP PC-48) (“PC-48”) to evaluate the ability of the surfactant blend to overcome the antagonistic incompatibility between each surfactant individually and bacterial spores. The test formulation comprised GCC and PC-48 at a total actives concentration of about 15.5 wt % and actives weight ratio of GCC to PC-48 of about 2.3:1. The test formulation also comprised 1 wt % alkyl sarcosinate surfactant (Maprosyl® 30-B) (0.3% actives), 0.2 wt % 2-phenoxyethanol, 2.68×10⁹ CFU/ml of a commercially available Bacillus spore blend, and water to total 100% by weight of the composition. The weight ratio of GCC to total anionic surfactant (PC-48 plus alkyl sarcosinate) was about 2.2:1.

Replicate test samples were prepared by diluting the test formulation in water in test jars to a dilution of 0.3 wt % and adding 1 wt % of a 33 g/L Nutrient Mix (30 g Tryptic Soy broth and 3.0 g/L Yeast Extract). The test samples were evaluated for bacterial germination and growth using the Sudan III dyed EVOO test described in Example 7, except that the test samples were placed on stir plates with a stir bar in each jar and stirred at a speed of 500 rpm, instead of using an orbital mixer. The test samples were photographed after 6 days of mixing, and showed significant haze, indicating that the surfactant blend in the formulation was able to overcome the incompatibility between the individual surfactants and the bacterial spores.

### Example 13: Structured Surfactant Systems with Alternative Surfactant Blends

Aqueous test compositions comprising different combinations of low HLB and high HLB incompatible surfactants were prepared to determine the ability of the surfactant combinations to form structured surfactant systems that can suspend the spores. The surfactant combinations tested were an ethoxylated alcohol (BIOSOFT® N91-2.5) as the low HLB surfactant in combination with either alpha sulfonated methyl esters (ALPHA-STEP® PC-48), alkyl ether sulfate (STEOL® CS-270C or CS-270 PLUS), or alkyl amine oxide (AMMONYX® LO) as the high HLB surfactant. Each test composition comprised the combination of low HLB and high HLB surfactants at a total actives concentration of about 16 wt %, but the relative weight ratio of the two surfactants was varied between 14:7 to 7:14. Each test composition also comprised 1 wt % alkyl sarcosinate surfactant (Maprosyl® 30-B) (0.3 wt % actives), 0.25 wt % 2-phenoxyethanol, and water to total 100% by weight of the composition. Testing showed that the combination of ethoxylated alcohol and amine oxide at product weight ratios in the range of about 14:7 to 10:13 ethoxylated alcohol to amine oxide could form structured surfactant systems that could suspend spores. The combination of ethoxylated alcohol with alpha sulfonated alkyl esters and the combination of ethoxylated alcohol with alkyl ether sulfate showed phase separation without creating a structured surfactant system.

The test compositions containing the ethoxylated alcohol and amine oxide surfactant combination in product weight ratios of 13.5:8, 12:10, and 10:13 were evaluated for yield stress as determined from an oscillation sweep measuring storage modulus and loss modulus as a function of oscillation stress. As used in this Example, yield stress is the crossover stress value where the loss modulus becomes higher than the storage modulus. The test compositions having surfactant weight ratios of 13.5:8 and 12:10 had yield stress values in the range of about 130 to about 170 Pa, whereas the composition having a surfactant weight ratio of 10:13 had a yield stress value of about 50 Pa. These results show that the compositions having surfactant weight ratios of 13.5:8 and 12:10 can provide yield stress values that could suspend spores.

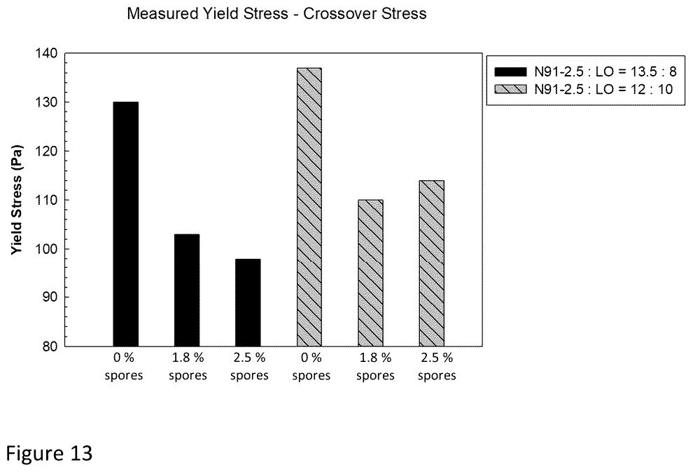

Different concentrations of spores were added to the test compositions comprising ethoxylated alcohol and amine oxide having weight ratios of 13.5:8 and 12:10 to determine the effect of spore addition on the yield stress of the compositions. The spore concentrations tested were 0 wt %, 1.8 wt %, and 2.5 wt % of a commercially available Bacillus spore blend containing 100×10⁹ CFU/ml spores. The test results are shown in the graph in FIG. 13. The yield stress graph in FIG. 13 shows that the yield stress of the compositions drops significantly when spores are added to the compositions. The results also show that higher values are obtained when the ethoxylated alcohol to amine oxide weight ratio is 12:10 compared to the 13.5:8 ratio, and that at the 12:10 weight ratio, the 2.5% spore concentration has a higher yield stress value than the 1.8% concentration.

The test compositions at the 13.5:8 and 12:10 surfactant weight ratios, and at spore concentrations of 1.8 wt % and 2.5 wt %, were visually evaluated for stability at two different temperatures. The temperatures tested were 25° C. and 50° C. At 25° C., the composition having a surfactant ratio of 12:10 provided a structured surfactant system that stably suspended spores at both the 1.8 wt % and 2.5 wt % concentrations, whereas the composition having the 13.5:8 ratio could only stably suspend the 1.8 wt % spore concentration. At 50° C., the composition having the 13.5:8 weight ratio was unstable at both spore concentrations and separated into layers, whereas the composition having the 12:10 ratio was still able to suspend the spores.

### Example 14: Compatibility of Ethoxylated Alcohol/Amine Oxide Surfactant Blend and Spores

Aqueous test compositions were prepared from a test formulation comprising the ethoxylated alcohol and alkyl amine oxide surfactant blend from Example 13 to evaluate the ability of the surfactant blend to overcome the antagonistic incompatibility between each surfactant individually and bacterial spores. The test formulation comprised the surfactant blend at a product weight ratio of ethoxylated alcohol to amine oxide of about 12:10, an actives weight ratio of ethoxylated alcohol to amine oxide of about 4.2:1, and a total actives concentration of about 16 wt %. The test formulation also comprised 1 wt % alkyl sarcosinate surfactant (Maprosyl® 30-B) (0.3% actives), 0.2 wt % 2-phenoxyethanol, 2.55×10⁹ CFU/ml of a commercially available Bacillus spore blend, and water to total 100% by weight of the composition. The actives weight ratio of anionic surfactant (alkyl sarcosinate) to amphoteric surfactant (alkyl amine oxide) was about 0.1:1.

The test compositions were prepared in duplicate at two different concentrations by diluting the test formulation in water in test jars to a dilution of 0.2 wt % and 1.0 wt %, and adding 1 wt % of a 33 g/L Nutrient Mix (30 g Tryptic Soy broth and 3.0 g/L Yeast Extract) to each test composition. The test compositions were evaluated for bacterial germination and growth using the Sudan III dyed EVOO test described in Example 7. The test compositions were photographed initially and again after 6 days of mixing. The test compositions at both concentrations showed significant haze, indicating that the surfactant blend in the formulation was able to overcome the incompatibility between the individual surfactants and the bacterial spores.

### Example 15: Diesel Fuel Digestion

This example examined the ability of Bacillus spores to germinate, grow, and digest diesel fuel when the spores are in an aqueous composition comprising a blend of incompatible surfactants. Replicate test samples were prepared by combining 48.3 g DI water, 1.0 g of the Table 3 formulation prepared with dialkyl amide, 1.0 g sodium xylene sulfonate (80% active), 0.2 g 33 g/L Nutrient Mix, and 0.5 g of a stock suspension of 10\hat{ }9 CFU/g of a commercially sourced mix of Bacillus spores. 2.0 g of Sudan III dyed (62.5 ppm) diesel fuel was added on top of the test samples. Replicate control samples were similarly prepared except that Sudan III dyed (62.5 ppm) EVOO was added on top of the control samples. The test and control samples were mixed at 500 rpm on identical stir plates for 15-30 minutes, allowed to phase separate, and then photographed. The samples were then stirred for 7 days, allowed to phase separate, and re-photographed. The test samples showed significant haze similar to that of the EVOO control samples. The results show that the mix of Bacillus spores in the test samples were able to germinate, grow, and digest the diesel fuel.

### Example 16: Preservative Study

A preservative screening study was performed to determine a sufficient amount of preservative to include in selected MEC formulations to provide efficacy against pathogenic bacteria and fungi. The base formulations used in the study were a formulation similar to Composition A from Example 5, but without the spores, and a formulation similar to the Example 14 formulation, but without the spores and 2-phenoxyethanol. Test formulations were prepared by adding different preservatives in different amounts to the base formulations. Each test formulation was tested for efficacy against pathogenic organisms using a preservative screening test. For the screening test, microorganisms are grown separately according to industry standards described in PCPC Test Method M-7 “A Rapid Method for Preservation Testing of Water-Miscible Personal Care Products”, and combined as an inoculum challenge prior to introduction to the formulation test sample. The inoculum is added to each test formulation for predetermined time points, and after appropriate nutrition, the test formulations are evaluated for remaining live count. Reduction of live count is calculated against a control, and a sample showing sufficient percent reduction is accepted as an effectively preserved formulation. Table 4 provides the details of the preservative and amount used in each test formulation and the results of the screen testing.

TABLE 4

| | | | | | | Fungal | | |
|---|---|---|---|---|---|---|---|---|
| | | Base | | Bacterial | Bacterial | 24 | Fungal | |
| Preservative | Active % | Formula | pH | 24 hours | 7 days | hours | 7 days | Result |
| 2-phenoxyethanol | 1.5% | Example 5 | 6.5-7.0 | <50% | <50% | <50% | <50% | Did not |
| | | | | | | | | pass |
| 2-phenoxyethanol | 1.5% | Example 14 | N/A | Not | 87.5% | Not | <50% | Did not |
| | | | | Tested | | Tested | | pass |
| 2-phenoxyethanol | 1.5% | Example 5 | 9.0 | 99.4% | >99.6% | 29.3% | <50% | Did not |
| | | | | | | | | pass |
| 2-phenoxyethanol | 1.5% | Example 14 | 9.0 | 98.4% | >99.6% | 75.0% | 95.6% | Did not |
| | | | | | | | | pass |
| 2-phenoxyethanol + | 1.5% | Example 5 | 5.0 | >99.6% | >99.6% | 99.2% | 99.6% | Pass |
| Benzoic Acid | | | | | | | | |
| 2-phenoxyethanol + | 1.5% | Example 14 | 5.0 | >99.6% | >99.6% | 99.6% | 99.6% | Pass |
| Benzoic Acid | | | | | | | | |
| Kathon-CG | 7.5 ppm | Example 5 | 6.5-7.5 | >99.6% | >99.6% | 99.6% | 99.6% | Pass |
| Kathon-CG | 7.5 ppm | Example 14 | 6.5-7.5 | >99.6% | >99.6% | 99.6% | 99.6% | Pass |
| Kathon-CG | 2.5 ppm | Example 14 | 6.5-7.5 | >99.6% | >99.6% | 99.6% | 99.6% | Pass |
| Acticide B20 | 400 ppm | Example 14 | 6.5-7.5 | >99.6% | >99.6% | 99.6% | 99.6% | Pass |
| Benzoic Acid | 1.0% | Example 14 | 5.0 | >99.6% | >99.6% | 99.6% | 99.6% | Pass |

The results show that 1.5 wt % 2-phenoxyethanol, even at a pH of 9.0, is not sufficient to provide efficacy against pathogenic bacteria and fungi in either base formulation. However, the combination of 2-phenoxyethanol and benzoic acid at 1.5 wt % (0.75 wt % each) passed the screening test for both base formulations. The results also show that the base formulations containing other preservatives known in the art passed the screening test for efficacy against pathogenic bacteria and fungi.

Additional embodiments of the invention are described in the following numbered paragraphs.

Paragraph 1. A composition for overcoming antagonistic surfactant incompatibility between a surfactant and a beneficial microorganism, the composition comprising about 2.0% to about 15% active weight sodium lauryl ether sulfate; 0% to about 2.0% by active weight alcohol alkoxylate; about 2.0% to about 15% by active weight cocoamidopropyl betaine; 0% to about 2% by active weight dimethyl lauramide/myristamide or lauryl lactyl lactate; 0% to about 7.0% active weight MgCl₂ or NaCl; at least 10⁴ CFU/mL of a live beneficial microorganism, and water to total 100% by weight of the composition.

Paragraph 2. The composition of Paragraph 1 wherein the alcohol alkoxylate is present in the composition in an amount of 0.1% to 2% by active weight, and comprises a linear alcohol ethoxylate.

Paragraph 3. The composition of Paragraph 2, wherein the linear alcohol ethoxylate has 8 moles of ethoxylate per molecule.

Paragraph 4. A composition for overcoming antagonistic surfactant incompatibility between a surfactant and a beneficial microorganism, the composition comprising about 0.1% to about 15% active weight sodium lauryl sulfate; 0.1% to about 15% by active weight lauramine oxide; at least 10⁴ CFU/mL of a live beneficial microorganism, and water to total 100% by weight of the composition.

Paragraph 5. The composition in any preceding Paragraph, further comprising one or more enzymes, preferably proteases, amylases, lipases, or combinations thereof.

Paragraph 6. A method for overcoming antagonistic surfactant incompatibility between a surfactant and a live beneficial microorganism, the method comprising (a) providing at least 10⁴ cfu/mL of a live beneficial microorganism; (b) providing from 0.1% to 15% by active weight, based on the total weight of the composition, of an anionic surfactant that, individually, exhibits antagonistic surfactant incompatibility when in contact with a live beneficial microorganism and adversely affects viability, growth, or metabolic activity of the live beneficial microorganism, wherein the anionic surfactant active is selected from the group consisting of alkyl sulfate, alkyl ether sulfate, alkyl sarcosinate, alpha sulfonated alkyl esters, alkyl glutamate, and combinations thereof; (c) providing 0.1% to 15% by active weight of an additional surfactant that, individually, exhibits antagonistic surfactant incompatibility when in contact with a live beneficial microorganism and adversely affects viability, growth, or metabolic activity of the live beneficial microorganism, wherein the additional surfactant active is selected from the group consisting of (i) amphoteric surfactants selected from the group consisting of alkyl amine oxide, alkyl betaine, alkyl amidopropyl betaine, alkyl sulfobetaine, and combinations thereof, and (ii) glyceryl monoesters and/or diesters; (d) optionally, providing a nonionic surfactant other than glyceryl monoesters and diesters that, individually, exhibits antagonistic surfactant incompatibility when in contact with a live beneficial microorganism and adversely affects viability, growth, or metabolic activity of the live beneficial microorganism; (e) mixing together the beneficial microorganism, the anionic surfactant, the additional surfactant, and optionally, the nonionic surfactant in a liquid carrier to form a composition; wherein the anionic surfactant, and the additional surfactant, and optionally, the nonionic surfactant, in combination, overcome the antagonistic surfactant incompatibility between the surfactants and the live beneficial microorganism, and facilitate viability, growth, and metabolic activity of the live beneficial microorganism.

Paragraph 7. The method of Paragraph 6, wherein the anionic surfactant is alkyl ether sulfate in an amount of 2.0% to 15% by active weight, the amphoteric surfactant is alkyl amidopropyl betaine in an amount of 2.0% to 15% by active weight, and the nonionic surfactant is alcohol ethoxylate in an amount of 0.1% to 2% by active weight.

Paragraph 8. The method of Paragraph 6 or Paragraph 7, further comprising mixing a dialkyl amide or an alkyl lactyl lactate in an amount of 0.1% to 2% by active weight with the anionic surfactant, the amphoteric surfactant, and the nonionic surfactant.

Paragraph 9. The method of any of Paragraphs 6-8, further comprising mixing MgCl₂ or NaCl in an amount of 0.1% to 7.0% by weight with the anionic surfactant, the amphoteric surfactant, and the nonionic surfactant.

Paragraph 10. The method of Paragraph 6, wherein the anionic surfactant is a combination of alpha sulfonated alkyl ester in an amount of about 2% to about 7% by active weight and alkyl sarcosinate in an amount of about 0.2% to about 2% by active weight, and the additional surfactant is glyceryl monoesters and/or diesters in an amount of about 5% to about 15% by active weight.

Paragraph 11. The method of Paragraph 6, wherein the anionic surfactant is alkyl sarcosinate in an amount of about 0.2% to about 2% by active weight, the amphoteric surfactant is alkyl amine oxide in an amount of 2% to about 5% by active weight, and the nonionic surfactant is an ethoxylated alcohol having an HLB value of less than 10 in an amount of about 8% to about 20%, alternatively about 8% to about 15% by active weight.

Paragraph 12. The embodiments in any preceding Paragraph, wherein the composition maintains the beneficial microorganism in suspension without separation for a period of at least one month.

The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further, the examples are provided to not be exhaustive but illustrative of several embodiments that fall within the scope of the claims. Any references noted in the detailed description section of the instant application are hereby incorporated by reference in their entireties, unless otherwise noted.

What is claimed is:

1. A composition for overcoming antagonistic surfactant incompatibility between a surfactant and a beneficial microorganism, wherein the antagonistic surfactant incompatibility adversely affects viability, growth, or metabolic activity of a live beneficial microorganism, the composition comprising:
(a) 0.1% to about 15% by active weight of at least one anionic surfactant that, individually, exhibits antagonistic surfactant incompatibility when in contact with a live beneficial microorganism and adversely affects viability, growth, or metabolic activity of the live beneficial microorganism, wherein the anionic surfactant is selected from the group consisting of alkyl sulfate, alkyl ether sulfate, alkyl sarcosinate, alpha sulfonated alkyl esters, alkyl glutamate, and combinations thereof;
(b) 0.1% to about 15% by active weight of at least one additional surfactant that, individually, exhibits antagonistic surfactant incompatibility when in contact with a live beneficial microorganism and adversely affects viability, growth, or metabolic activity of the live beneficial microorganism, wherein the at least one additional surfactant is selected from the group consisting of (i) amphoteric surfactants and (ii) glyceryl monoesters and/or diesters;
(c) optionally, at least one nonionic surfactant other than glyceryl monoesters and diesters that, individually, exhibits antagonistic surfactant incompatibility when in contact with a live beneficial microorganism and adversely affects viability, growth, or metabolic activity of the live beneficial microorganism;
(d) at least 10⁴ CFU/mL of a live beneficial microorganism, wherein the live beneficial microorganism is at least one bacteria, yeast, or mold; and
(e) water to total 100% by weight of the composition,
wherein, the anionic surfactant, the at least one additional surfactant, and optionally, the nonionic surfactant, in combination in the composition overcome the antagonistic surfactant incompatibility between the surfactants and the live beneficial microorganism, and facilitate viability, growth, and metabolic activity of the live beneficial microorganism.

2. The composition of claim 1, wherein the amphoteric surfactant is one or more of alkyl amine oxide, alkyl betaine, alkyl amidopropyl betaine, or alkyl sulfobetaine.

3. The composition of claim 1, wherein the nonionic surfactant is one or more alcohol alkoxylates.

4. The composition of claim 1, wherein the at least one additional surfactant is an amphoteric surfactant, and the anionic surfactant and the amphoteric surfactant are present in the composition in a weight ratio of anionic surfactant to amphoteric surfactant of 5:1 to 1:2.

5. The composition of claim 1, wherein the composition further comprises a dialkyl amide or an alkyl lactyl lactate.

6. The composition of claim 1, wherein the composition comprises:
about 2.0% to about 15% active weight alkyl ether sulfate as the anionic surfactant;
0% to about 2.0% by active weight alcohol alkoxylate;
about 2.0% to about 15% by active weight alkylamidopropyl betaine as the amphoteric surfactant;
0% to about 2% by active weight dialkyl amide or alkyl lactyl lactate; and
0% to about 7.0% active weight MgCl₂ or NaCl.

7. The composition of claim 1, wherein the composition comprises:
an alkyl sarcosinate surfactant as the anionic surfactant, and alkyl betaine as the amphoteric surfactant.

8. The composition of claim 1, wherein the composition comprises alkyl sulfate as the anionic surfactant, and alkyl amine oxide as the amphoteric surfactant.

9. The composition of claim 1, wherein the at least one additional surfactant is glyceryl monoesters and diesters, and the composition comprises alpha sulfonated alkyl esters and alkyl sarcosinate as the anionic surfactants.

10. The composition of claim 1, wherein the composition comprises alkyl sarcosinate as the anionic surfactant, amine oxide as the amphoteric surfactant, and ethoxylated fatty alcohol as the nonionic surfactant.

11. The composition of claim 1, wherein the live beneficial microorganism comprises spores, preferably bacterial spores, and more preferably bacterial spores of Bacillus amyloliquefaciens, Bacillus brevis, non-pathogenic variants of Bacillus cereus, such as toyoi, Bacillus circulans, Bacillus clausii, Bacillus coagulans, Bacillus firmus, Bacillus halodurans, Bacillus lentus, Bacillus licheniformis, Bacillus megaterium, Bacillus methylotrophicus, Bacillus mycoides, Bacillus pasteurii, Bacillus, polyfermenticus, Bacillus polymyxa, Bacillus pumilus, Bacillus simplex, Bacillus sphaericus, Bacillus stearothermophilus, Bacillus subtilis, Bacillus thiaminolyticus, or Bacillus thuringiensis, or combinations thereof.

12. The composition of claim 1, wherein the live beneficial microorganism is in an amount of 1×10⁷ to 1×10¹⁰, preferably 1×10⁹ to 5×10⁹ CFU/g.

13. The composition of claim 1, wherein the composition has a viscosity of about 8,000 cP to about 30,000 cP at 6 rpm and ambient temperature, and a viscosity of about 3,000 cP to about 8,000 cP at 60 rpm and ambient temperature.

14. A method for overcoming antagonistic surfactant incompatibility between a surfactant and a live beneficial microorganism, wherein the antagonistic surfactant incompatibility adversely affects viability, growth, or metabolic activity of a live beneficial microorganism, the method comprising:
(a) providing at least 10⁴ cfu/mL of a live beneficial microorganism, wherein the live beneficial microorganism is at least one bacteria, fungi, or yeast;
(b) providing from 0.1% to 15% by active weight of an anionic surfactant that, individually, exhibits antagonistic surfactant incompatibility when in contact with a live beneficial microorganism and adversely affects viability, growth, or metabolic activity of the live beneficial microorganism, wherein the anionic surfactant is selected from the group consisting of alkyl sulfate, alkyl ether sulfate, alkyl sarcosinate, alpha sulfonated alkyl esters, alkyl glutamate, and combinations thereof;
(c) providing 0.1% to 15% by active weight of an additional surfactant that, individually, exhibits antagonistic surfactant incompatibility when in contact with a live beneficial microorganism and adversely affects viability, growth, or metabolic activity of the live beneficial microorganism, wherein the additional surfactant is selected from the group consisting of (i) amphoteric surfactants, and (ii) glyceryl monoesters and/or diesters;
(d) optionally, providing a nonionic surfactant other than glyceryl monoesters and diesters that, individually, exhibits antagonistic surfactant incompatibility when in contact with a live beneficial microorganism and adversely affects viability, growth, or metabolic activity of the live beneficial microorganism;
(e) mixing together the beneficial microorganism, the anionic surfactant, the additional surfactant, and optionally, the nonionic surfactant in a liquid carrier to form a composition;
wherein the anionic surfactant, and the additional surfactant, and optionally, the nonionic surfactant, in combination, overcome the antagonistic surfactant incompatibility between the surfactants and the live beneficial microorganism, and facilitate viability, growth, and metabolic activity of the live beneficial microorganism.

15. The method of claim 14, wherein the amphoteric surfactant is one or more of alkyl amine oxide, alkyl betaine, alkyl amidopropyl betaine, or alkyl sulfobetaine.

16. The method of claim 14, wherein the nonionic surfactant is one or more alcohol alkoxylates.

17. The method of claim 15, wherein the anionic surfactant and the amphoteric surfactant are present in the composition in an active weight ratio of anionic surfactant to amphoteric surfactant of 5:1 to 1:2.

18. The method of claim 14, wherein the composition further comprises a dialkyl amide or an alkyl lactyl lactate.

19. The method of claim 14 wherein the live beneficial microorganism comprises spores, preferably bacterial spores, and more preferably bacterial spores of Bacillus amyloliquefaciens, Bacillus brevis, non-pathogenic variants of Bacillus cereus, such as toyoi, Bacillus circulans, Bacillus clausii, Bacillus coagulans, Bacillus firmus, Bacillus halodurans, Bacillus lentus, Bacillus licheniformis, Bacillus megaterium, Bacillus methylotrophicus, Bacillus mycoides, Bacillus pasteurii, Bacillus, polyfermenticus, Bacillus polymyxa, Bacillus pumilus, Bacillus simplex, Bacillus sphaericus, Bacillus stearothermophilus, Bacillus subtilis, Bacillus thiaminolyticus, or Bacillus thuringiensis, or combinations thereof.

20. The method of claim 14, wherein the composition has a viscosity of about 8,000 cP to about 30,000 cP at 6 rpm and ambient temperature, and a viscosity of about 3,000 cP to about 8,000 cP at 60 rpm and ambient temperature.
